(19) United States
(12) Patent Application Publication
(10) Pub. No.: US 2015/0005510 A1
(43) Pub. Date: Jan. 1, 2015

(54) METHOD FOR CONVERSION OF DIAMMONIUM SUCCINATE IN FERMENTATION BROTH TO 2-PYRROLIDONE AND N-METHYLPYRROLIDONE

(71) Applicants: Thidarat Tosukhowong, Billerica, MA (US); Kirk Roffi, Reading, MA (US); Santosh Raghu More, Newark, NJ (US); Robert L. Augustine, Livingston, NJ (US); Setrak Tanielyan, Maplewood, NJ (US)

(72) Inventors: Thidarat Tosukhowong, Billerica, MA (US); Kirk Roffi, Reading, MA (US); Santosh Raghu More, Newark, NJ (US); Robert L. Augustine, Livingston, NJ (US); Setrak Tanielyan, Maplewood, NJ (US)

(73) Assignee: MYRIANT CORPORATION, Quincy, MA (US)

(21) Appl. No.: 14/241,532

(22) PCT Filed: Aug. 31, 2012
(86) PCT No.: PCT/US2012/053543

§ 371 Date: Feb. 27, 2014

Related U.S. Application Data
(60) Provisional application No. 61/573,207, filed on Sep. 1, 2011.

(51) Int. Cl. C07D 207/404; C07D 207/267

(52) U.S. Cl. CPC C07D 207/404; C07D 207/267
USPC 548/545; 548/543

(57) ABSTRACT

This invention relates to a process for preparing 2-pyrrolidone (also called 2-pyrrolidinone) and N-methylpyrrolidone (also called N-methylpyrrolidinone) from diammonium succinate in fermentation broth. In the first stage of this invention, renewable carbon resources are utilized to produce diammonium succinate through biological fermentation. In the second stage of this present invention, diammonium succinate is converted into 2-pyrrolidone and N-methylpyrrolidone through a two step reaction. Both the steps of the reaction leading to the production of 2-pyrrolidone and N-methylpyrrolidone are carried out in a solvent phase to prevent the loss of succinimide through hydrolysis.

### CROSS-REFERENCE TO RELATED APPLICATION

This application claims the priority of the U.S. Provisional Application Ser. No. 61/573,207, filed on Sep. 1, 2011.

### BACKGROUND OF THE INVENTION

2-Pyrrolidone and N-methylpyrrolidone are useful industrial chemicals. N-methylpyrrolidone is currently used as an industrial solvent. It is a highly stable aprotic polar solvent, which is miscible with water. The global production capacity of N-methylpyrrolidone was 226 million pounds in 2006. It is widely used as a solvent in electronic process, polyurethane processing, coating, or as a replacement for methylene chloride in paint strippers. In butadiene recovery process, N-methylpyrrolidinone is also used as an extractive distillation solvent.

2-pyrrolidone is a very good high-boiling polar solvent, which has a wide variety of applications in pharmaceuticals and intermediates. For example, 2-pyrrolidone is used as plasticizer and coalescing agent for coating application. Most of the 2-pyrrolidone production is converted into n-vinylpyrrolidone monomer, which is then polymerized to make polyvinylpyrrolidone polymer (PVP or Povidone). PVP has many applications, such as binding agent, film former, and emulsion stabilizer. This compound is water soluble and has a very good tackifying property. In consumer product and cosmetics industry, PVP is widely used as ingredients in shampoo, hairspray, oral rinse, ophthalmic composition, etc. Furthermore, this compound is FDA approved and can be used as a binder in pharmaceutical tablets. The global production of PVP in 2008 was around 110 million pounds.

Currently, the typical process to make 2-pyrrolidone and N-methylpyrrolidone involves a reaction between gamma-butyrolactone (GBL) with ammonia and methylamine, respectively. GBL is currently a co-product in the hydrogenation process to produce 1,4-butanediol (BDO). There are several chemical routes to synthesize BDO, but one of the most economical routes is starting from butane as a raw material. First, butane is oxidized to produce maleic anhydride. Then, maleic anhydride can be converted to BDO via the BP/Lurgi Geminox process or the Davy Technology process. The former process recovers maleic anhydride as maleic acid and performs liquid-phase hydrogenation to produce a mixture of BDO with tetrahydrofuran (THF) and/or GBL. In the Davy process, maleic anhydride is esterified to dimethyl maleate, which is then vaporized and fed to a vapor-phase hydrogenation system to produce dimethyl succinate. Dimethyl succinate undergoes hydrogenolysis reaction to produce GBL and BDO, which can be further converted into THF. These products are separated by distillation and methanol is recycled back to the esterification reactor. The reaction steps of this process are shown in FIG. 1.

To make 2-pyrrolidone and N-methylpyrrolidone, GBL is reacted with ammonia gas and methylamine, respectively. The overall petroleum-based process to derive 2-pyrrolidone via the Davy process is depicted in FIG. 2.

The conventional process of producing 2-pyrrolidone and N-methylpyrrolidone via butane or benzene oxidation to maleic anhydride is not a sustainable process, since the raw material is derived from petroleum. One of the possible pathways to derive a bio-based GBL is by esterifying the bio-succinic acid to dialkyl succinate, followed by a hydrogenation step to produce BDO, THF, and GBL. The present invention provides a novel route to directly convert diammonium succinate present in the fermentation broth to 2-pyrrolidones via succinimide in order to reduce the overall energy consumption and carbon footprint compared to the conventional multi-step process. The biological process to make bio-succinic acid is also CO₂ negative, since E. coli strain producing succinic acid through fermentation process requires about 0.5 mole of CO₂ to make each mole of succinic acid. Furthermore, during the conversion of diammonium succinate to succinimide, ammonia will be removed and can then be recycled back to the fermentation process. Thus the production of bio-based 2-pyrrolidone and N-methylpyrrolidone will help expand the portfolio for the value-added green chemicals.

There are existing literatures related to a process to convert succinic acid or diammonium succinate to pyrrolidones. U.S. Pat. No. 3,198,808 discloses a process to produce pyrrolidones from a liquid-phase reaction of ammonia and diacids, such as succinic acid, maleic acid, or fumaric acid. Water and/or organic solvent such as dioxane and THF can be used as a solvent medium for the reaction. Catalysts were chosen from metal oxides of Co, Ni and mixture thereof. The examples in this U.S. patent showed that the reaction yield to pyrrolidone is in the range of 75-84%.

U.S. Pat. No. 3,448,118 suggested a process for preparing n-alkyl-2-pyrrolidone from a reaction between succinic acid and primary amine in a single step reaction at 200-300° C. and at least 50 bars of H₂ pressure. The maximum yield for N-methylpyrrolidone was found to be 81.8%.

Frye et al (2005) have reported the conversion of succinate to GBL, BDO, THF, and pyrrolidones. The catalysis work has been conducted using both reagent-grade succinic acid, as well as fermentation-derived feedstocks.

Using reagent grade raw material, Frey et al (2005) conducted several reactions in a semi-batch process. The hydrogenation results starting from succinic acid, ammonia, and methanol gave a maximum yield to pyrrolidones over 80% at 265° C. with the mole ratio of succinic acid/NH₃/methanol=1.0/2.0/2.0 using Rh-based catalyst. Reduction in the molar ratio of methanol does not significantly affect the overall yield, but it increases the selectivity to 2-pyrrolidone over N-methylpyrrolidone. Higher temperature also increased the yield to pyrrolidones. Furthermore, reduction in ammonia reduced the total yield to pyrrolidones.

When the reagent-grade N-methylsuccinimide is used as the raw material, the hydrogenation reaction yielded a higher selectivity to N-methylpyrrolidone, especially when the temperature is reduced from 265° C. to 200° C. The overall yield to pyrrolidones as high as 89% is achieved.

Frey et al (2005) studied the methylation reaction to synthesize N-methylsuccinimide. When using succinic acid with ammonia and methanol as reactants, the maximum yield of 83.3% n-methylsuccinimide is obtained at 300° C. When succinimide and methanol are reacted together, N-methylsuccinimide can be synthesized with a high yield of 87.5% in 0.5 hrs, but the yield decreases to 82.3% after 2.5 hrs. These results confirm that N-methylpyrrolidone can be synthesized from diammonium succinate via the formation of N-methylsuccinimide as an intermediate compound.

Frey et al (2005) has also performed hydrogenation of reagent-grade N-methylsuccinimide in a continuous flow trickle bed reactor packed with Rh/Re catalyst. The conversion of N-methylsuccinimide was above 88% at the temperature above 200° C. The yield to N-methylpyrrolidone was the highest at the highest test temperature of 250° C., which contradicts the results from the semi-batch reactor test where the highest yield was obtained at the lower temperature of 200° C. The maximum yield to N-methylpyrrolidone in the continuous flow reactor is 67% with a very low amount of 2-pyrrolidone. Compositions of other by-products were not shown in this case.

Frey et al (2005) has also tested the fermentation-derived succinic acid that had been processed through some cleanup steps. However, they found the conversion rates to be an order of magnitude lower than that of the reagent-grade succinic acid.

US Patent Application Publication No. US 2010/0044626 disclosed a process to convert succinates from fermentation broth to pyrrolidones. The main processing step requires the removal of ammonia and/or water in the fermentation broth by distillation. Subsequently, the remaining bottom product is distilled to form succinimide or alkylsuccinimide. This patent application Publication suggested that succinimide is further reacted without further isolation or intermediate purification to pyrrolidones. The invention also taught that the risk of catalyst poisoning by secondary constituents of the fermentation broth is lowered by the distillative purification step. In Example 2.1 of this patent application Publication, 1030 g of fermentation broth consisting of 13 g/l of diammonium succinate was supplemented with 58.5 g of synthetic diammonium succinate. (The ratio of synthetic diammonium succinate:bio-based diammonium succinate was calculated to be about 4.5:1). This supplemented diammonium succinate was distilled at 175° C. to remove water and was converted to succinimide at 250° C. After that, distillation was performed and the overhead product contained 88% succinimide. The yield was not shown in this example.

In example 2.3, 1284 g solution of 5 gmol synthetic diammonium succinate was prepared by mixing 684 g of 25% aqueous ammonia and approximately 600 g of succinic acid (47 wt % succinic acid and 13 wt % ammonia in water). This solution was used as reactant. The mixture was reacted at 250° C. at standard pressure. Then, the mixture was distilled at reduced pressure to produce 486 g of distillate with 92 wt % of succinimide. From this data, the reaction yield to succinimide is calculated to be 90%.

In example 3.1, hydrogenation reaction of succinimide to 2-pyrrolidone was performed in an autoclave. The yield was found to be 95% after 24 hrs. However, the temperature, pressure, and catalyst used in this test were not explained.

Finally, this patent application Publication tested a vapor-phase alkylation reaction between 2-pyrrolidone and methanol in a reactor packed with 80% Al₂O₃/20% SiO₂ catalysts. Using a 1:1 mixture of 2-pyrrolidone and methanol, the reaction yield to N-methylpyrrolidone was found to be 48.1% and 61.3% N-methylpyrrolidone at 300° C. and 350° C., respectively.

To our best knowledge, there has not been any successful conversion of diammonium succinate in the fermentation broth to 2-pyrrolidone and N-methylpyrrolidone. Reagent-grade diammonium succinate or fermentation broth supplemented with four-fold excess of synthetic diammonium succinate does not represent fermentation-derived diammonium succinate solution.

By developing a process for making derivatives from bio-succinic acid, consumers will have more alternatives for chemicals derived from lower CO₂-intensity process.

### BRIEF SUMMARY OF THE INVENTION

This present invention provides a process for the manufacturing of 2-pyrrolidone and N-methylpyrrolidone from diammonium succinate in the fermentation broth obtained by fermenting renewable carbon sources using biocatalyst with the ability to produce succinic acid. The fermentation is conducted at a neutral pH by means of using ammonium hydroxide as a neutralizing agent leading to the accumulation of diammonium succinate in the fermentation broth. The fermentation broth containing diammonium succinate is centrifuged to remove the cell debris followed by ultrafiltration step to remove protein contaminants. The sugars and amino acids in the fermentation broth are removed by means of subjecting the fermentation broth through an adsorption process. In one embodiment, the fermentation broth is preferably concentrated and then subjected to a thermochemical conversion process to produce succinimide. In a preferred embodiment, the concentrated fermentation broth is subjected to activated carbon treatment before subjecting it to thermochemical conversion process to produce succinimide. In a most preferred embodiment, the thermochemcial conversion process is carried out in a solvent environment to prevent the production of succinamic acid from the hydrolysis of succinimide. The succinimide resulting from thermochemical conversion process is subjected to hydrogenation in the presence of a hydrogenation catalyst to produce 2-pyrrolidone. Again, in the preferred aspect of the present invention, the hydrogenation of succinimide is carried out in the presence of a solvent to prevent the hydrolysis of succinimide and to enhance the production of 2-pyrrolidone.

In another embodiment of the present invention, the thermochemical conversion of ammonium succinate is carried out in the presence of an alkylating agent such as methanol to produce N-methylsuccinimide. The N-methyl succinimide resulting from the thermochemical conversion process is subjected to hydrogenation reaction in the presence of a metal catalyst. In a preferred embodiment, both the alkylation reaction leading to the production of N-methyl succinimide and the conversion of N-methylsuccinimide to N-methylpyrrolidone are carried out in a solvent environment to prevent the hydrolysis of N-methylsuccinimide and thereby increase the production of N-methylpyrrolidone.

In another embodiment of the present invention, the succinimide derived from the thermochemcial conversion of ammonium succinate is subjected to hydrogenation reaction involving a metal catalyst in the presence of an alkylating reagent such as methanol leading to the production of N-methylpyrrolidone. In a preferred aspect of the present invention the combined hydrogenation and alkylation reactions using succinimide as the reactant leading to the production of N-methylpyrrolidone is carried out in a solvent environment to prevent the hydrolysis of succinimide and to increase the production of N-methylpyrrolidone. Alternatively, n-methylsuccinimide is produced by reacting concentrated fermentation broth with methanol. Subsequently, n-methylsuccinimide is purified by solvent extraction. The solvent extracted n-methylsuccinimide is hydrogenated to produce n-methylpyrrolidone in the presence of hydrogenation catalyst. The 2-pyrrolidone and N-methylpyrrolidone are recovered through distillation process.

### BRIEF DESCRIPTION OF THE DRAWINGS

The following figures are included to illustrate certain aspects of the present invention, and should not be viewed as exclusive embodiments. The subject matter disclosed is capable of considerable modifications, alterations, combinations, and equivalents in form and function, as will occur to those skilled in the art and having the benefit of this disclosure.

FIG. 1. Reaction steps in the Davy's process for producing 1,4-butanediol and tetrahydrofuran from N-butane.

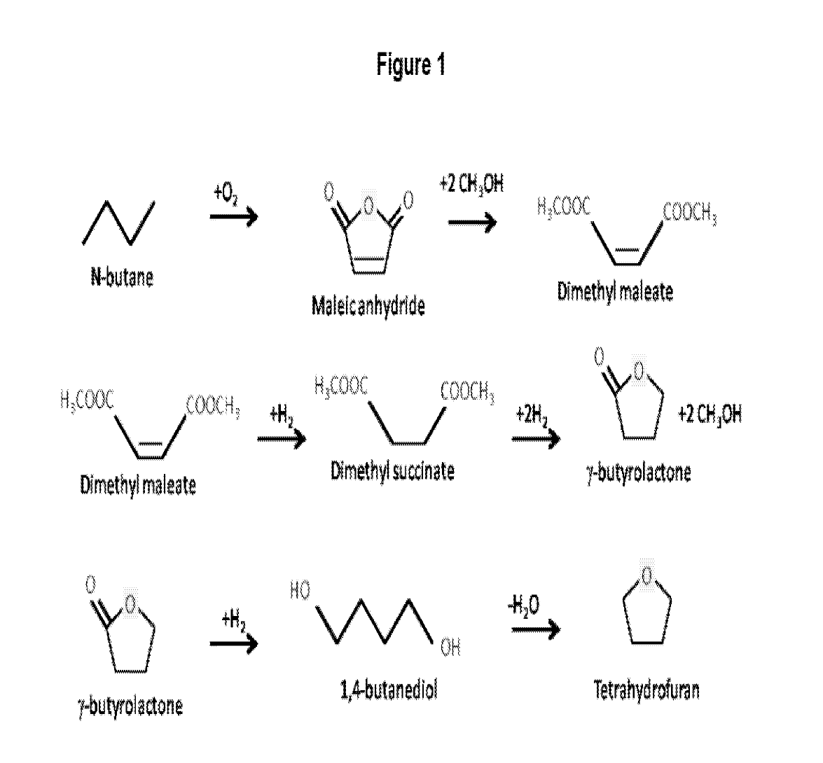

FIG. 2. Conventional process based on butane to make 2-pyrrolidone via maleic anhydride and gamma-butyrolactone.

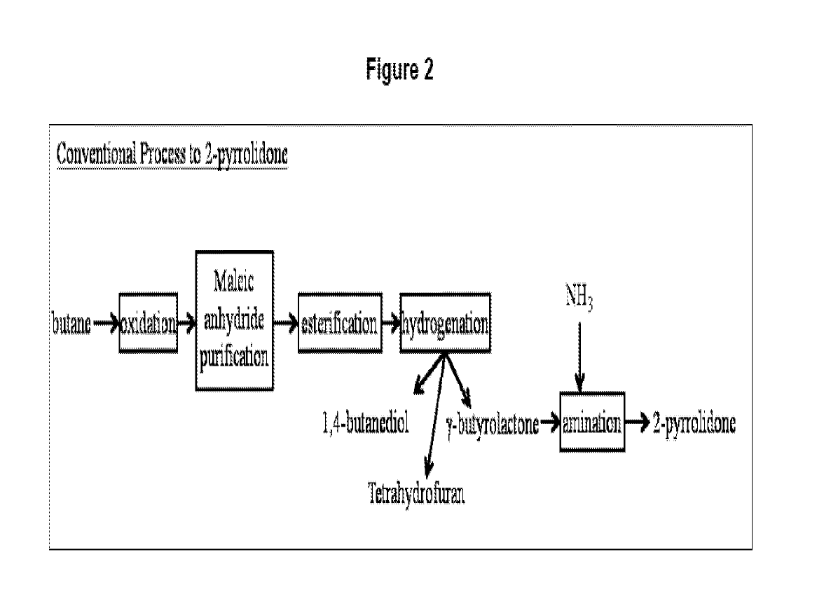

FIG. 3. Process schematic for producing 2-pyrrolidone and other derivative chemicals from bio-based crystalline succinic acid. Crystalline succinic acid recovered from ammonium succinate present in the fermentation broth is subjected to esterification reaction followed by hydrogenation reaction to produce □-butyrolactone which in turn is subjected to amination reaction to produce 2-pyrrolidone.

FIG. 4. Reaction pathway from diammonium succinate to 2-pyrrolidone. When the fermentation broth containing diammonium succinate is subjected to elevated temperature, the diammonium succinate is believed to be converted either into monoammonium succinate or succindiamide. Monoammonium succinate can further be converted either into succinamic acid or succinimide. Succindiamide can be converted into succinimide. There is also an interconversion between succinamic acid and succinimide. Upon hydrogenation both succinamic acid and succinimide can produce 2-pyrrolidone while the hydrogenation of succinimide and succinamic acid in the presence of methanol produces N-methylpyrrolidone.

FIG. 5. Simplified process schematic for direct conversion of diammonium succinate to 2-pyrrolidone. Fermentation of biomass-derived sugars in a mineral medium with appropriate biocatalysts in the presence of ammonia and carbon dioxide results in the accumulation of diammonium succinate in the fermentation broth. The diammonium succinate recovered from the fermentation broth is subjected to a thermochemical reaction leading to the formation of succindiamide and then succinimide with a release of ammonia which can be recovered and recycled in the fermentation process. The succinimide from thermochemical reaction upon hydrogenation yields 2-pyrrolidone.

FIG. 6. A process for direct conversion of diammonium succinate containing fermentation broth to 2-pyrrolidone. Fermentation broth containing ammonium succinate purified through cell separation, ultrafiltration, adsorption and concentration steps is subjected to thermochemical reaction in an imide reactor. The products from thermochemical reaction are subjected to solvent extraction with an organic solvent. The aqueous phase containing succindiamide is recycled back to imide reactor. The organic phase containing succinimide is subjected to hydrogenation reaction to produce 2-pyrrolidone, which is recovered by distillation and the organic solvent is recycled.

FIG. 7. A process for direct conversion of diammonium succinate containing fermentation broth to N-methylpyrrolidone. Fermentation broth containing ammonium succinate purified through cell separation, ultrafiltration, adsorption and concentration steps is subjected to thermochemical reaction in an imide reactor. The products from thermochemical reaction are subjected to solvent extraction with an organic solvent. The aqueous phase containing succindiamide is recycled back to imide reactor. The organic phase containing succinimide is subjected to hydrogenation reaction in the presence of methanol to produce N-methylpyrrolidone, which is recovered by distillation and the organic solvent is recycled.

FIG. 8. A process for direct conversion of diammonium succinate containing fermentation broth to N-methylpyrrolidone via N-methylsuccinimide. Fermentation broth containing ammonium succinate purified through cell separation, ultrafiltration, adsorption and concentration steps is subjected to thermochemical reaction in an imide reactor in the presence of methanol. The products from thermochemical reaction are subjected to solvent extraction with an organic solvent. The aqueous phase containing succindiamide is recycled back to imide reactor. The organic phase containing N-methylsuccinimide is subjected to hydrogenation reaction to produce N-methylpyrrolidone, which is recovered by distillation and the organic solvent is recycled.

FIG. 9. A process for the conversion of diammonium succinate in the fermentation broth to 2-pyrrolidone according to preferred embodiment of the present invention. Water in the fermentation broth is replaced with organic solvent prior to subjecting the solution to thermochemical conversion process in order to prevent the hydrolysis of the succinimide to succinamic acid. Succinimide is subjected to hydrogenation reaction in the presence of a suitable metal catalyst to produce 2-pyrrolidone.

FIG. 10. A process for the conversion of diammonium succinate in the fermentation broth to N-methylpyrrolidone according to preferred embodiment of the present invention. Water in the fermentation broth is replaced with organic solvent prior to subjecting the solution to thermochemical conversion process in order to prevent the hydrolysis of succinimide to succinamic acid. The succinimide thus produced is subjected to hydrogenation reaction in the presence of suitable metal catalyst and methanol to produce N-methylpyrrolidone.

FIG. 11. A process for the conversion of diammonium succinate in the fermentation broth to N-methylpyrrolidone according to preferred embodiment of the present invention. Water in the fermentation broth is replaced with organic solvent and the solution is subjected to thermochemical conversion process in the presence of methanol leading to the production of N-methylsuccinimide. In the next stage, N-methylsuccinimide is subjected to hydrogenation reaction to produce N-methylpyrrolidone.

FIG. 12. Comparison of fermentation broth obtained from the fermenter and the concentrated fermentation broth. The fermentation broth obtained directly from the fermenter had succinic acid at the concentration of 70 grams/L. The concentration of fermentation broth through evaporation in a rotary evaporator resulted in the succinic acid concentration of 210 g/L accompanied by the development of a dark coloration.

FIG. 13. Effect of activated carbon treatment on the color of the concentrated fermentation broth. The concentrated fermentation broth was treated with activated carbon as described in the specification and the activated carbon was removed through centrifugation. With increasing concentration of activated carbon used, the color of the concentrated fermentation broth was totally removed and the broth became a colorless liquid. The tube in the extreme left contains fermentation broth which was not subjected to any activated carbon treatment. The second tube from the left contains fermentation broth treated with 0.99% (w/w) activated carbon. The tube in the middle contains fermentation broth treated with 2.9% (w/w) activated carbon. The tube second form the right contains fermentation broth treated with 4.7% (w/w) activated carbon. The tube at the extreme right contains fermentation broth treated with 9.1% (w/w) activated carbon.

FIG. 14. Organization of the Parr Reactor used in the hydrogenation reaction to produce 2-pyrrolidone. The various components of the Parr Reactor are described in detail in the sections below.

### DETAILED DESCRIPTION OF THE PREFERRED EMBODIMENTS

In general, the present invention relates to the process for producing derivative chemicals from dicarboxylic acid. Dicarboxylic acid suitable for the present invention is preferably derived from biomass through fermentation process. The dicarboxylic acid suitable for the present invention can be represented by Formula (A).

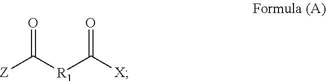

where Z and X independently represent one or more C, H, O, N, S, a halide, and a counter-ion. Z and X can also be O⁻; the O⁻ may be free or with a counter ion. The counter ion can be either NH4⁺ or Na⁺ or K⁺. R1 is a linear or branched, saturated or unsaturated hydrocarbon or substituted hydrocarbon. Preferably R1 contains 1 to 10 carbon atoms.

Compound of Formula (A) is taken up in a solvent having a boiling point higher than that of water and subjected to a thermochemical conversion in the presence or absence of an alkylating agent to produce a compound of Formula (B).

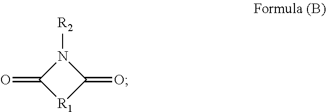

Where R1 is linear or branched, saturated or unsaturated hydrocarbon or substituted hydrocarbon. Preferable R1 contains 1 to 10 carbon atoms. R2 can be an alkyl (linear, cyclic or branched, saturated or unsaturated), a substituted alkyl group, an aromatic group or hydrogen.

Compound of formula (B) is subjected to catalytic carbonyl reduction reaction in the presence of a metal catalyst to produce desirable compounds. The preferred embodiment of the present invention relates to methods for preparing bio-based 2-pyrrolidone and/or N-methylpyrrolidone from diammonium succinate derived from biomass through fermentation process as described below.

The present invention provides for, in at least some embodiments, reaction pathways utilizing chemical reactions and catalysts that effectively (i.e., with higher conversion percentages) and selectively produce 2-pyrrolidone and N-methylpyrrolidone. As illustrated further herein, surprisingly, succinimide, the substrate for the production of 2-pyrrolidone and N-methylpyrrolidone, is produced more effectively in the presence of a high-boiling polar organic solvent. Consequently, reaction pathways and catalysts described herein may, in some embodiments, provide for cost-effective, environmentally friendly industrial scale production of 2-pyrrolidone and N-methylpyrrolidone.

A high-boiling polar solvent is referred as “solvent’ in the present invention. The boiling point of the solvent of the present invention is higher than that of water.

It should be noted that when “about” is used herein at the beginning of a numerical list, “about” modifies each number of the numerical list. It should be noted that in some numerical listings of ranges, some lower limits listed may be greater than some upper limits listed. One skilled in the art will recognize that the selected subset will require the selection of an upper limit in excess of the selected lower limit.

As used herein, the term “reaction pathway” refers to the reaction or series of reactions for converting reactants to products that comprise 2-pyrrolidone and N-methylpyrrolidone. In some embodiments, a reaction pathway of the present invention may comprise a step at elevated temperatures. In some embodiments, a reaction pathway of the present invention may further comprise a catalytic reaction.

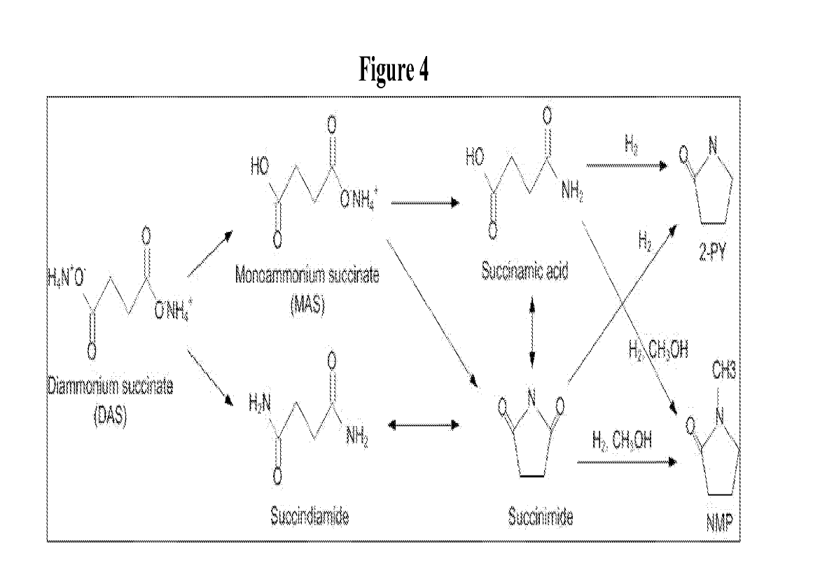

The reaction pathway of the present invention is illustrated in FIG. 4, using diammonium succinate as the reactant. The reaction pathway has two separate and distinct steps. In the first step of the pathway, diammonium succinate in the concentrated fermentation broth is subject to thermochemical reaction in the presence of a solvent having a boiling point higher than that of water. This thermochemical reaction is initiated during or after the removal of the water from fermentation broth through evaporation. When the fermentation broth containing ammonium succinate is used there is neither a need to obtain the free succinic acid nor a need to add any exogenous ammonium. In fact, the ammonium released during the process of removing water through evaporation and subsequent thermochemical reaction phase can be captured using appropriate methods and the ammonia thus recovered can be recycled to the fermentation process for maintaining the neutral pH inside the fermentor during the production of succinic acid. On the other hand, when succinic acid is obtained as sodium or potassium salt in the fermentation broth, it is desirable to obtain free succinic acid to enter into the reaction pathway for the production of 2-pyrrolidone and N-methylpyrrolidone. Moreover, when succinic acid recovered from a fermentation broth containing sodium or potassium salt of succinic acid is used as a reactant, it is necessary to add additional ammonium in the initial thermochemical reaction step to achieve the formation of succinimide.

In the second step of the reaction pathway, the product from the first step of the reaction pathway is subjected to catalytic carbonyl reduction reaction to produce desirable products. The preferred embodiment of the present invention relates to methods for preparing bio-based 2-pyrrolidone and/or N-methylpyrrolidone from diammonium succinate derived from biomass through fermentation process as described below.

Reactants suitable for use in conjunction with reaction pathways of the present invention include all those compounds that can be represented by Formula (A). In a preferred embodiment, the reactants suitable for the present invention can be represented by salts of dicarboxylic acids, including but not limited to the salts of succinic acid. Optionally the salts of dicarboxylic acid suitable for the present invention can be derived from a group consisting of ammonium succinate, potassium succinate and sodium succinate, either one of which or all of which may be derived from biomass materials.

Reactants suitable for use in conjunction with reaction pathways of the present invention may be produced by any known means. In some embodiments, reactants may be biologically-derived, chemically-derived, or a combination thereof. Examples of biologically-derived reactants may be found in the following international patent applications published under Patent Cooperation Treaty: WO2008/115958, WO2011/115067, WO2011/063055, WO2011/063157, WO2011/082378, WO2011/123154, WO2011/130725, WO2012/018699 and WO2012/082720, all of which are incorporated herein by reference.

The fermentation process for producing dicarboxylic acid may, in some embodiments, be a batch process, a continuous process, or a hybrid process thereof. A large number of carbohydrate materials derived from natural resources can be used as a feedstock in conjunction with the fermentative production of dicarboxylic acids described herein. For example, sucrose from cane and beet, glucose, whey containing lactose, maltose and dextrose from hydrolyzed starch, glycerol from biodiesel industry, and combinations thereof may be suitable for the fermentative production of dicarboxylic acids described herein. Microorganisms may also be created with the ability to use pentose sugars derived from hydrolysis of cellulosic biomass in the production of dicarboxylic acids described herein. In some embodiments, a microorganism with ability to utilize both 6-carbon containing sugars such as glucose and 5-carbon containing sugars such as xylose simultaneously in the production of dicarboxylic acid is a preferred biocatalyst in the fermentative production of dicarboxylic acids. In some embodiments, hydrolysate derived from cheaply available cellulosic material contains both C-5 carbon and C-6 carbon containing sugars and a biocatalyst capable of utilizing simultaneously C-5 and C-6 carbon containing sugars in the production of dicarboxylic acid is highly preferred from the point of producing low-cost dicarboxylic acid suitable for the conversion into 2-pyrrolidone and N-methylpyrrolidone.

In some embodiments, the fermentation broth may be utilized at various points of production, e.g., after various unit operations have occurred like filtration, acidification, polishing, concentration, or having been processed by more than one of the aforementioned unit operations. In some embodiments, when the fermentation broth may contain about 6 to about 15% dicarboxylic acid on weight/weight (w/w) basis, the dicarboxylic acid may be recovered in a concentrated form. The recovery of dicarboxylic acid in a concentrated form from a fermentation broth may be achieved by a plurality of methods and/or a combination of methods known in the art.

During the fermentation methods described herein, at least one alkali material (e.g., NaOH, CaCO₃, (NH₄)₂CO₃. NH₄HCO₃, NH₄OH, or any combination thereof) may be utilized in order to maintain the near neutral pH of the growth medium. Addition of alkali materials to the fermentation broth often results in the accumulation of dicarboxylic acid in the form of inorganic salts. In some embodiments, ammonium hydroxide may be a preferred alkali material for maintaining the neutral pH of the fermentation broth. With the addition of ammonium hydroxide to the fermentation medium for the production of succinic acid, ammonium succinate may accumulate in the fermentation broth. Because ammonium succinate has higher solubility in aqueous solution, it may have an increased concentration in the fermentation broth. One way to obtain succinic acid from the fermentation broth containing ammonium succinate may include micro and ultra filtering the fermentation broth followed by ion exchange chromatography. The sample coming out of ion exchange chromatography may, in some embodiments, then be subjected to conventional electrodialysis to obtain succinic acid in the form of a concentrated free acid. For the purpose of present invention, the ammonium succinate in the fermentation broth may be used after micro filtration and ultrafiltration steps without the need for producing free succinic acid. However, when potassium hydroxide or sodium hydroxide is used as a neutralizing agent in the fermentation broth leading to the production of potassium succinate or sodium succinate, it is necessary to obtain the free succinic acid before entering into the reaction pathway for the production of 2-pyrrolidone and N-methylpyrrolidone.

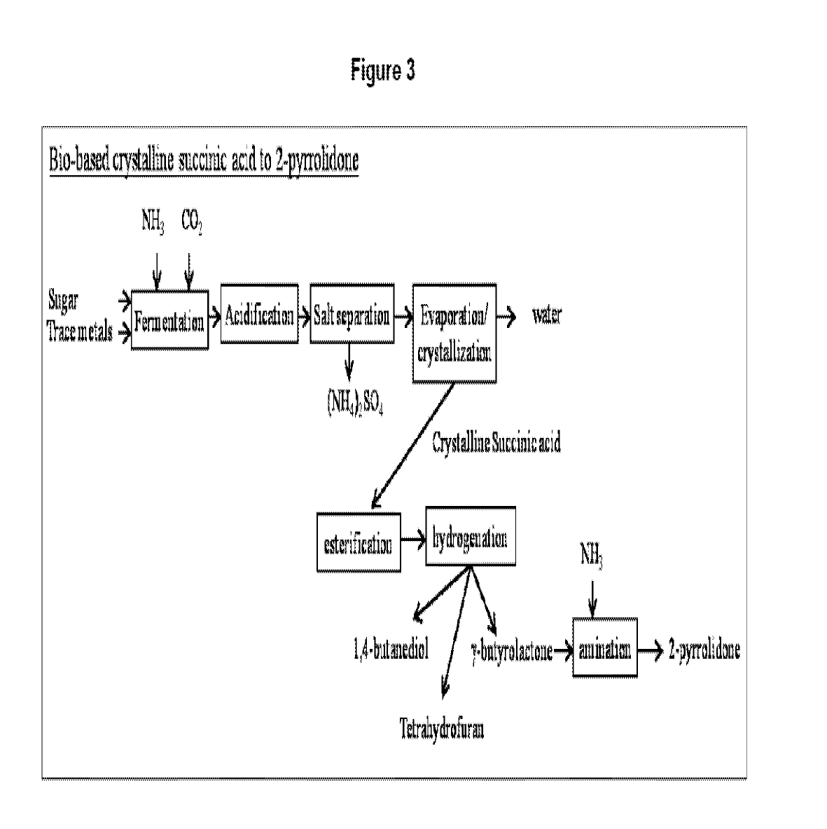

One could use two different approaches for the production of bio-based 2-pyrrolidone and N-methylpyrrolidone. Under one approach according to the present invention, bio-based 2-pyrrolidone and N-methylpyrrolidone are derived from bio-based crystalline succinic acid purified from the fermentation broth containing diammonium succinate. The bio-based crystalline succinic acid can be used as a drop-in replacement for maleic anhydride or maleic acid to produce 1,4-BDO, THF, and GBL. The process to produce bio-based 2-pyrrolidone via crystalline succinic acid is depicted in FIG. 3. In this process succinic acid is separated and purified from the fermentation broth using methods well known in the art. Shown in FIG. 3 are the steps involved in the separation of succinic acid from fermentation broth using the steps of centrifugation, filtration, salt separation, ion exchange polishing and evaporation/crystallization steps. The highly pure crystalline succinic acid thus obtained is esterified to make dimethyl succinate. Subsequently, dimethyl succinate can be hydrogenated to produce GBL, BDO, and THF. The resulting bio-based GBL is a raw material to make pyrrolidones.

In the biological fermentation process using E. coli to produce succinic acid, inorganic alkali and trace nutrient chemicals are added to the fermenter to maintain the condition where the organisms can function optimally. For example, E. coli strain KJ122 obtained through genetic manipulations produces succinic acid at the highest yield when the pH is around 6.5-7.0. As a result, bases, such as potassium hydroxide, ammonium hydroxide, are added to maintain the pH during the course of the fermentation. At the end of the fermentation process, the organic acid products are in the form of salts of the carboxylic acids. Thus when ammonium hydroxide is used as the neutralizing base in the fermentation process involving KJ122 strain of E. coli, succinic acid accumulates at the end of fermentation in the form of diammonium succinate along with ammonium acetate. The fermentation broth is clarified to remove cell mass and protein via centrifugation and ultrafiltration step. To convert the dilute solution of ammonium succinate to succinic acid, there needs to be a step to provide proton to the broth. This can be achieved by an acidification step (e.g. with sulfuric acid) or an ion-exchange step. Succinic acid needs to be separated from ammonium sulfate and the remaining solution, which primarily contains water and other impurities such as unconverted sugars, amino acids, and inorganic nutrients. There are several technologies that can be used to separate ammonium sulfate from succinic acid, such as via a continuous chromatography, a continuous ion exchange process, or a solvent extraction method.

Optionally amino acids, remaining cations, and anions, as well as color bodies can be further removed by an ion-exchange and a color adsorption system downstream of the salt splitting step to produce high-purity succinic acid. A simple approach to remove color bodies form the fermentation broth is to use activated carbon. The fermentation broth can be treated with activated carbon for specific period of time and the activated carbon can be separated from the fermentation broth to completely remove the color bodies from the fermentation broth. Finally, the purified succinic acid solution is sent to the evaporator and the crystallizer to produce white crystalline succinic acid.

In another embodiment of the present invention, 2-pyrrolidone is produced from fermentation broth containing ammonium succinate. While it is feasible to produce pyrrolidones from bio-based crystalline succinic acid, the direct conversion of ammonium succinate in the fermentation broth to 2-pyrrolidones will significantly improve the overall economics and reduce the energy consumption and the waste generation of the overall process as it eliminates major processes in the production of the succinic acid crystals such as salt splitting, polishing and crystallization.

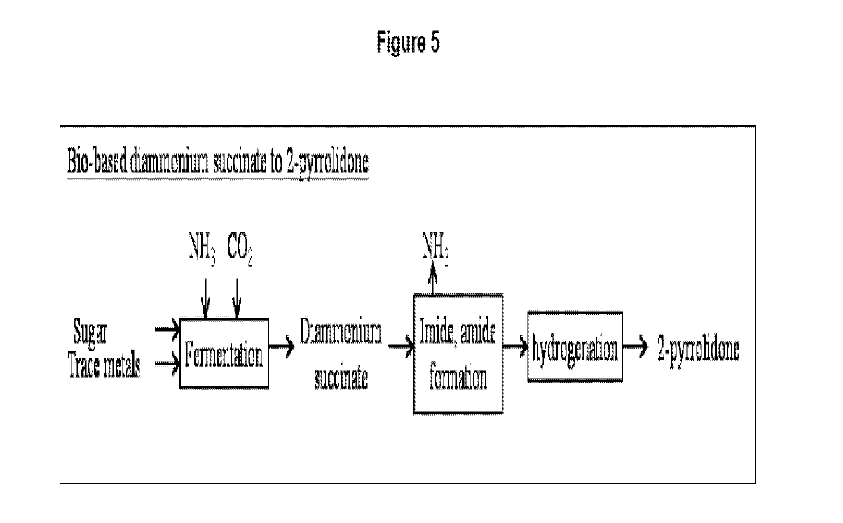

In this new paradigm, diammonium succinate in the fermentation broth will be used to produce succinimide in the first step, and then 2-pyrrolidone in the second step. During the ring closure step, ammonia can be recovered and recycled back to the fermenter. 2-pyrrolidone is produced via hydrogenation of succinimide, while N-methylpyrrolidone can be produced via hydrogenation of succinimide in the presence of methanol. Alternatively, N-methylpyrrolidone can be produced via hydrogenation of N-methylsuccinimide, which is a product of the reaction of succinimide with methanol. In another aspect of the present invention, as illustrated in FIG. 4, monoammonium succinate derived from diammonium succinate is converted into succinamic acid. Succinamic acid can be hydrogenated to produce 2-pyrrolidone. When the hydrogenation of succinamic acid is carried out in the presence of methanol, N-methyl pyrrolidone is obtained. There is equilibrium between succinamic acid and succinimide. The succinimide upon hydrolysis yields succinamic acid. Thus when the succinimide is present in an aqueous environment, it is converted into succinamic acid through hydrolysis. One disadvantage with the presence of succinamic acid is that it tends to polymerize and a polymerization of succinamic acid tends to reduce the yield of final products namely 2-pyrrolidone and N-methylpyrrolidone. The present invention provides a method to prevent the hydrolysis of succinimide to succinamic acid. According to the present invention, the hydrolysis of succinimide can be prevented by means of replacing the water in the fermentation broth with a polar solvent having a boiling point higher than that of water (aprotic, oxygen containing solvents). Such solvents include, but not limited to, diglyme, triglyme, tetraglyme, propylene glycol, dimethylsulfoxide (DMSO), dimethylformamide (DMF), dimethylacetamide, dimethylsulfone, sulfolane, polyethylene glycol (PEG), butoxytriglycol, N-methylpyrrolidone, (NMP), 2-pyrrolidone, gammabutyrolactone, dioxane, methyl isobutyl ketone (MIBK) and the like. The reaction pathway from diammonium succinate to 2-pyrrolidone is depicted in FIG. 4. The overall process for producing 2-pyrrolidone according to the preferred embodiment of the present invention is shown in FIG. 5.

The diammonium succinate concentration in the fermentation broth derived from a fermentation run with an efficient succinic acid biocatalyst is about 100 g/L. This high level of diammonium succinate concentration is accompanied by various impurities that can be of concern to hydrogenation catalysts, including residual sugars, amino acids, anions, and cations. When the source of sugars comes from biomass, there tend to be higher concentrations of impurities. If these impurities are not removed prior to heating the diammonium succinate containing broth to form succinimide, sugar and amino acid can undergo a Maillard reaction to form high-molecular weight compounds that may harm the catalyst or complicate the downstream purification. Furthermore, some ions can potentially form complex with the hydrogenation catalyst resulting in the catalyst deactivation, while chloride can cause corrosion problem to the equipment at high temperature. In order to overcome the poor catalytic conversion efficiency and selectivity, the impurities in the fermentation broth can be removed using the techniques well-known in the art such as adsorption/ion exchange technology. Moreover, the fermentation broth can further be concentrated before subjecting it to catalytic reaction to yield succinimide. Succinimide can be further purified to remove amino acids and other impurity prior to hydrogenation via an extraction or a solvent replacement process. Finally, a number of hydrogenation catalysts and operating parameters can be screened to obtain the highest yields to 2-pyrrolidone and N-methylpyrrolidone.

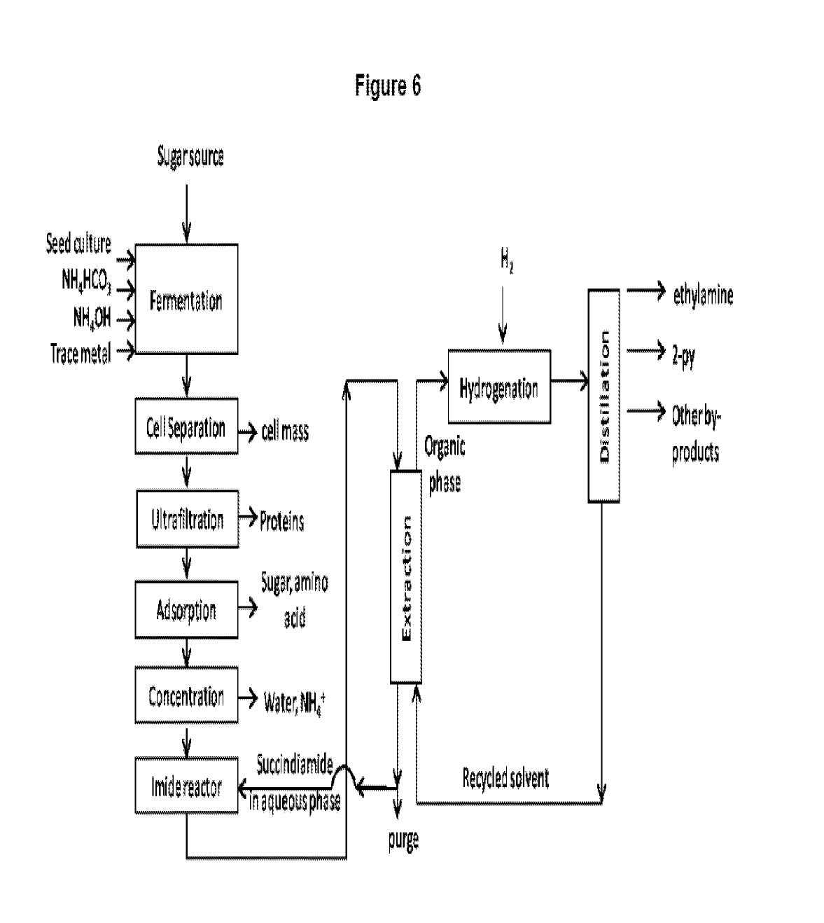

The proposed processes to directly convert diammonium succinate containing fermentation broth to 2-pyrrolildone and N-methylpyrrolidone are outlined in FIGS. 6-8.

In succinic acid fermentation process, sugar syrup and CO₂ source are fed to the fermenter in a fed-batch manner under anaerobic condition. As E. coli produces succinic acid, the pH has to be controlled to the near neutral range by gradually feeding ammonium hydroxide solution into the fermenter. After the production rate of succinic acid slows down to near 0 g/L-hr, the fermenter is discharged. Typically, impurity found in the fermentation broth includes acetic acid, amino acids, and residual sugars. Cell mass is removed from fermentation broth via a solid separation method, such as by centrifugation or microfiltration. Then, proteins should be removed by ultrafiltration in order to avoid further side reactions from protein degradation products.

Prior to the reaction to form succinimide under high temperature, it is important to remove as much residual sugars, amino acids, and anions as possible to avoid formation of Maillard reaction products. This can be achieved by an adsorption process, such as by using activated carbon. Then, the solution can be concentrated to remove water in order to improve the kinetics of the reaction. Water removal can be achieved by following a number of techniques well known in the art. In a preferred embodiment the water removal leading to the concentration of fermentation broth is achieved by using reverse osmosis (RO). Normally, the fresh water used in medium preparation for the fermentation has to be filtered by the RO unit. By means of using reverse osmosis to concentrate the fermentation broth, it is possible to obtain a water fraction that is suitable to meet the requirement for water in the preparation of fermentation broth. The RO permeate stream may contain ammonium ions, but that should be suitable for reuse in the NH₄OH preparation tank associated with fermentation unit. Furthermore, when reverse osmosis is used in the concentration of fermentation broth, the energy requirement is substantially reduced when compared to the distillation process.

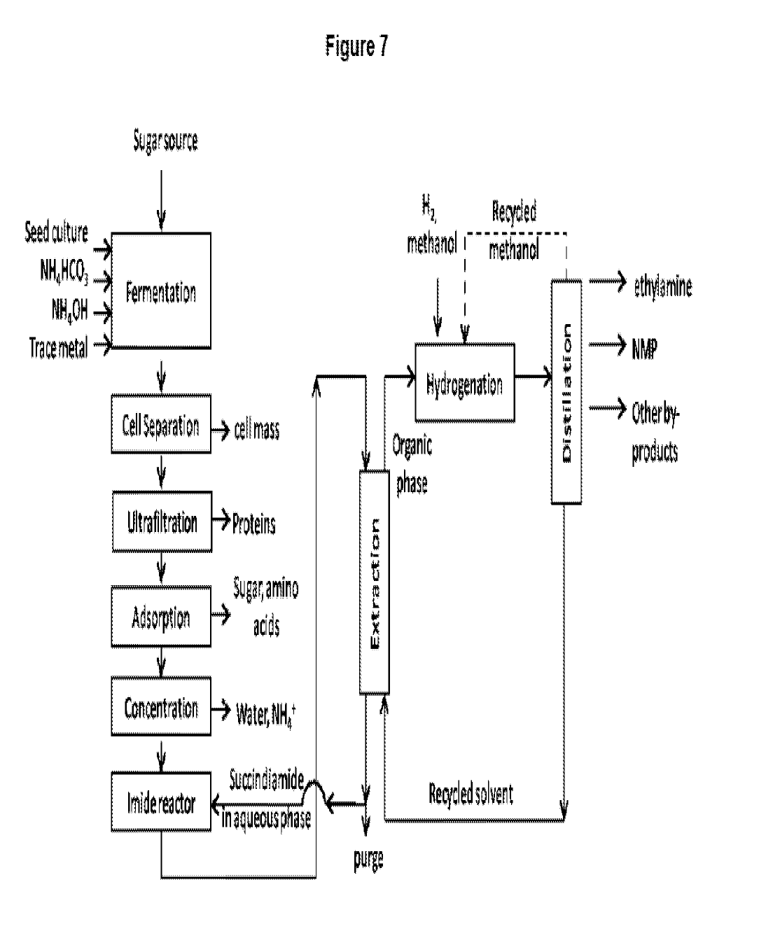

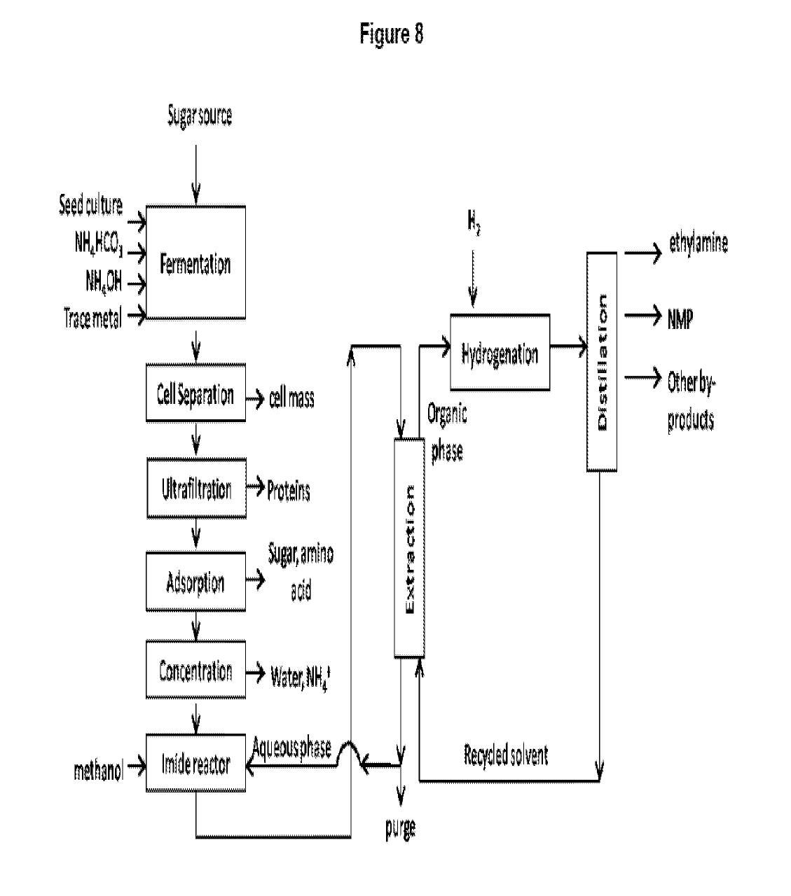

After the fermentation broth containing diammonium succinate has been concentrated, it is sent to a reactor to form a mixture of succinimide, succinamic acid, and succindiamide under high temperature. During this step any remaining by-products in the fermentation broth containing diammonium succinate may also undergo side reactions. For example, ammonium acetate can be converted into acetamide and aspartic acid can become aspargine. Subsequently, ionic impurity can be removed from succinimide and acetamide by solvent extraction method. Succindiamide, which has very low solubility in organic solvents, is likely to stay in the aqueous phase and can be recycled back to the reactor to be further converted into succinimide. Amino acids and remaining sugars (if any) also have low solubility in organic solvent, so they will remain in the aqueous phase with succindiamide. Periodically, impurity can be purged to reduce the buildup in the process. The organic phase from the extractor containing succinimide and acetamide is sent to the catalytic hydrogenation reactor to produce 2-pyrrolidone and ethylamine, respectively (FIG. 6). In the process of producing N-methylpyrrolidone from extracted succinimide, methanol is added to the hydrogenation reactor so that methylation and hydrogenation take place in the same reactor (FIG. 7). Alternatively, N-methylsuccinimide can be produced by contacting methanol to the concentrated ammonium succinate broth at elevated temperature. Ammonium acetate in the fermentation broth may also react to form N-methylacetamide in this step. Subsequently, N-methylsuccinimide is purified by extraction and then hydrogenated to yield N-methylpyrrolidone (FIG. 8). Finally, N-methylpyrrolidone from the hydrogenation reactor can be purified by distillation.

In a preferred embodiment of the present invention, in order to reduce the hydrolysis of succinimide, the water in the fermentation broth is replaced with a polar solvent having a boiling point higher than that of water. A suitable organic solvent in appropriate volume is added to the fermentation broth after cell separation, ultrafiltration, concentration and adsorption steps and the temperature of the resulting fermentation broth is increased to the level that would allow the water in the fermentation broth to evaporate. Once the water in the fermentation broth is fully evaporated, the temperature is increased to the level that would allow the thermochemical conversion of diammonium succinate to succinimide. This thermochemical conversion can be achieved in the temperature range of 100-300° C. and preferably in the temperature range of 120-180° C. and most preferably in the temperature range of 140-160° C.

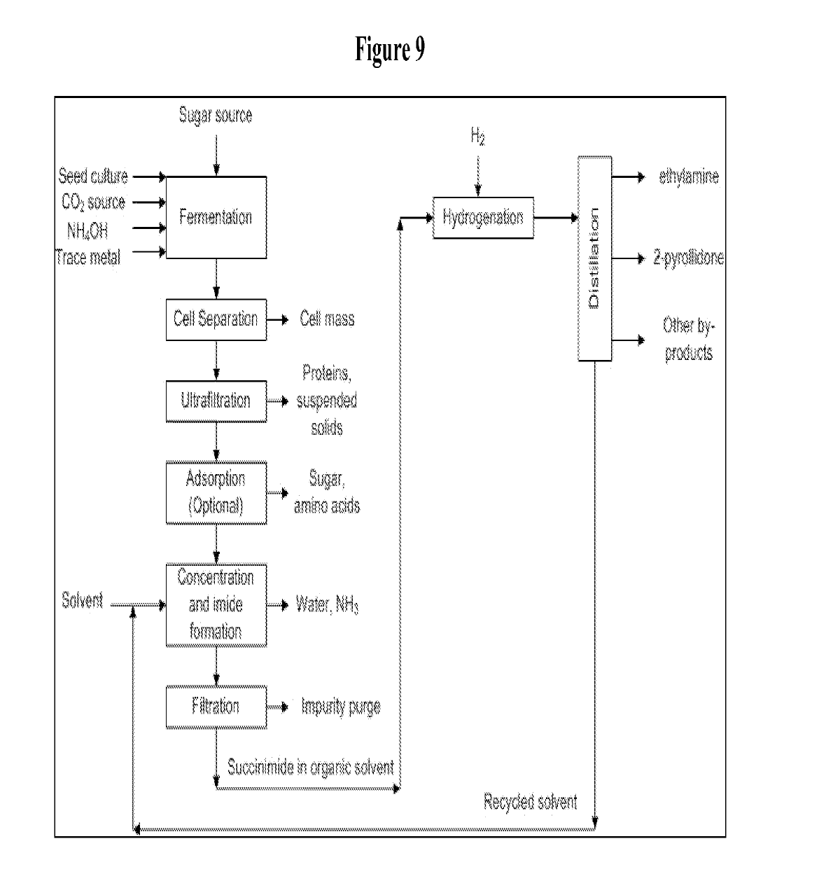

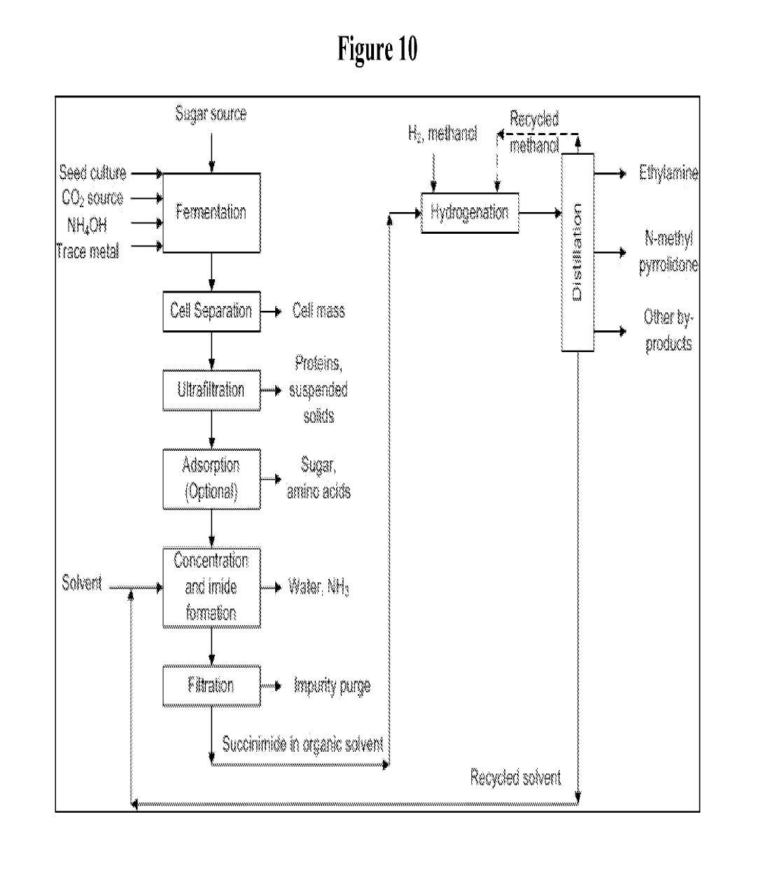

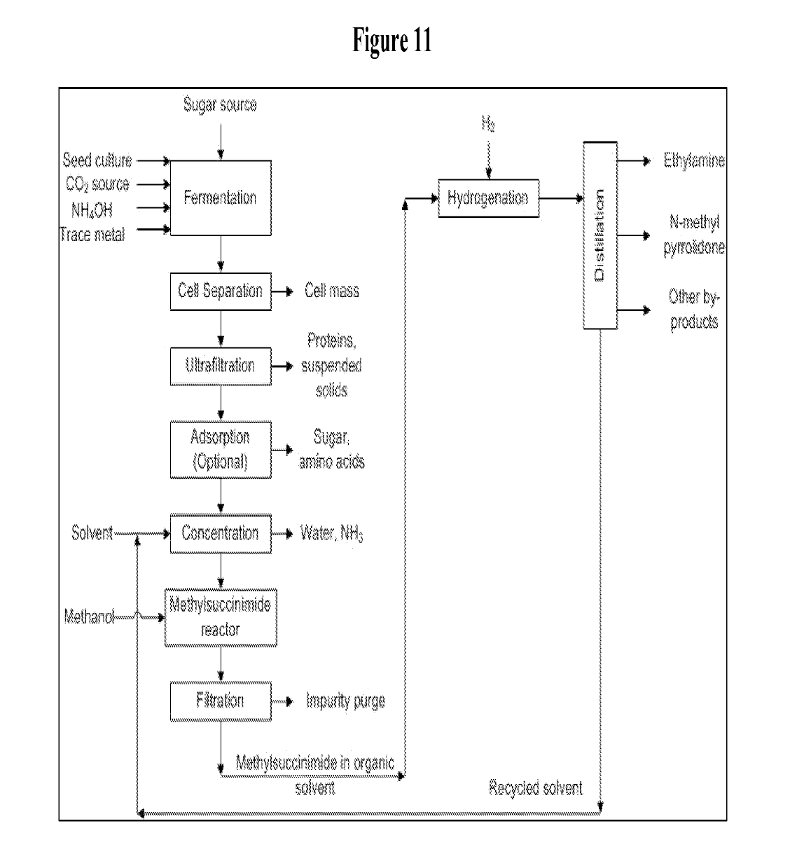

Three different aspects of the preferred embodiment of the present invention are illustrated in the FIGS. 9-11. As shown in FIG. 9, in one aspect of the preferred embodiment of the present invention, an organic solvent is used to replace water in the fermentation broth before the initiation of the thermochemical conversion process to produce succinimide. The succinimide thus produced is subject to carbonyl reduction reaction in the presence of a suitable metal catalyst to produce 2-pyrrolidone (FIG. 9). In another aspect of the preferred embodiment of the present invention as illustrated in FIG. 10, after the formation of succinimide in the solvent-replaced fermentation broth, the hydrogenation reaction is carried out in the presence of suitable metal catalyst and methanol leading to the production of N-methylpyrrolidone in place of 2-pyrrolidone. In yet another aspect of the preferred embodiment of the present invention, the solvent-replaced fermentation broth is subjected to thermochemical conversion process at the elevated temperature in the presence of methanol leading to the production of N-methylsuccinimide in place of succinimide (FIG. 11).

The present invention relates to the manufacture of biomass derived 2-pyrrolidone and N-methyl pyrrolidone. The present invention discloses (1) a process for producing 2-pyrrolidone from biomass-derived diammonium succinate present in the fermentation broth and (2) a process for producing N-methylpyrrolidone from biomass-derived diammonium succinate in the fermentation broth.

In the manufacture of 2-pyrrolidone using fermentation broth containing diammonium succinate, the thermochemical conversion of diammonium succinate into succinimide is followed by a catalyst-mediated carbonyl reduction process leading to the production of 2-pyrrolidone.

The manufacture of N-methylpyrrolidone using fermentation broth containing diammonium succinate can be carried out in two different ways. According to one method of the present invention, the diammonium succinate is subjected to thermochemical conversion leading to the production of succinimide which is subsequently subjected to a catalytic carbonyl reduction reaction in the presence of methanol resulting in the production of N-methylpyrrolidone. According to the other method of the present invention, in the first stage, the diammonium succinate in the fermentation broth is subjected to both thermochemical reaction and alkylation reaction simultaneously leading to the production of N-methyl succinimide. In the second stage of the process, the N-methyl succinimide produced in the first stage is subjected to catalyst-mediated carbonyl reduction process in the presence of hydrogen leading to the production of N-methylpyrrolidone.

Hydrogenation of succinimide to produce 2-pyrrolidone involves a catalyzed carbonyl reduction process and is carried out in the presence of hydrogen. A catalyzed carbonyl reduction process leading to the production of N-methylpyrrolidone using succinimide as the substrate is carried out in the presence of hydrogen and methanol. A catalyzed carbonyl reduction process can also be followed to hydrogenate N-methylsuccinimide to produce N-methylpyrrolidone.

The conversion efficiency and selectivity of each of the process steps according to the present invention are influenced by a number of factors. For example, the cyclization and alkylation reactions are influenced by the amount of water present in the reaction medium, the temperature of the reaction vessel and the ammonium concentration. The term ammonium as used in the present invention includes both NH3 and NH4⁺. Where succinate is provided in a non-ammonia form, ammonia is added to the reaction mixture to carry out the first stage of the reaction pathway where succinic acid is converted into succinimide. The ammonia to succinic acid ratio can be adjusted to achieve the maximum yield for the intermediate product succinimide as well as a maximum yield for the final products such as 2-pyrrolidone and N-methylpyrrolidone. The ammonia to succinic acid ratio is preferably held at or less than 2:1 in the reaction mixture. Similarly the carbonyl reduction can be influenced by the type of the catalyst used, temperature of the reaction, hydrogen pressure and the amount of water present in the medium.

The hydrogenation catalyst useful in the present invention contains one or more metals selected from a group consisting of Fe, Ni, Pd, Pt, Co, Sn, Rh, Re, Ir, Os, Au, Ru, Zr, Ag, and Cu. The catalyst may contain more than one metal element like Pd—Re or Rh—Re combination. Additionally, the catalyst may comprise a support. The support for the catalyst may comprise a porous carbon support, a metallic support, a metallic oxide support or mixtures thereof.

### EXPERIMENTAL SECTION

### General Remarks

Table 1 provides formulas for several calculations used throughout the Example section.

A plurality of catalysts was used in the present invention. The catalysts used in the examples presented herein were obtained from W.R. Grace Company, Johnson Matthey, Alfa Aesar, and Evonik (Table 2).

### Reaction Protocols

Analytical HPLC Procedure.

This procedure describes the conditions used with a High Pressure Liquid Chromatography (HPLC) apparatus to identify and quantify succinic acid and its derivatives obtained through using various reaction processes according to the present invention. The procedure below is described using succinic acid. A person skilled in the art of using HPLC will modify this procedure to quantify the other chemical compounds that are involved in the present invention.

Known quantity of solid succinic acid crystal (0.50 grams) is dissolved in 100 ml of 0.008N sulfuric acid, mixed well, filtered through a 0.2 □m syringe filter and used as a standard in calibrating the HPLC apparatus. Liquid experimental samples containing succinic acid is diluted 10× in 0.008N sulfuric acid. Smaller dilution may be used for experimental samples with trace amounts of succinic acid. The diluted experimental samples containing succinic acid is filtered through a 0.2 □m syringe filter and stored in a HPLC vial for analysis. Agilent 1200 HPLC apparatus is used with BioRad Aminex HPX-87H and BioRad Microguard Cation H⁺ (Guard Column). Column temperature was maintained at 50° C. and the flow rate was kept at 0.6 mL/minute. UV 210 nm and RI detectors are used. Under this operational condition the following elution times are observed: succinic acid=12.1 minutes; succinamic acid=17 minutes; succinimide=18 minutes; succindiamide=35 minutes.

Determination of Cation Concentration in Succinic Acid Samples Using Ion Chromatography System (ICS).

Dionex 1100 ion chromatography system with Dionex CSRS 300 (4 mm) suppressor, Dionex IonPac CS16-HC column and Dionex IonPac CG16-HC guard column is used for the determination of ammonium, sodium and potassium concentration in the succinic acid samples. 28 mM methanesulfonic acid is used as eluent and is prepared in the following way. Approximately 1000 ml of high purity water is added to a 2000 ml volumetric flask, 5.76 g of concentrated methanesulfonic acid solution is transferred to the water in the flask, the volume is brought to 2000 ml with water, mixed well by inversion and transferred to the eluent bottle on the ICS. A multi-element cation standard is useful to generate calibration curves. At least three different calibration standards (20, 10 and 1 ppm) are used to establish a calibration curve for each ion. Liquid samples for analysis are diluted using deionized water and filtered through a 0.2 □m filter. Solid samples are dissolved in appropriate volumes of deionized water and filtered through 0.2 □m filter. The following parameters are used in running the ICS. Flow rate: 1.5 mL/minute; column temperature: 40° C.; Cell temperature: 45° C.; Suppressor current: 88 mA; Sample delivery speed: 4 ml/minute.

Determination of Anion Concentration in Succinic Acid Samples Using Ion Chromatography System (ICS).

Dionex 1100 ion chromatography system with Dionex CSRS 300 (4 mm) suppressor, Dionex IonPac CS16-HC column and Dionex IonPac CG16-HC guard column is used for the determination of chloride, sulfate, and phosphate concentrations in succinic acid samples. Standards should have a known purity in order to accurately calculate the anion concentrations in the sample. Using concentrated standards, working standards are prepared. For example, by means of dissolving 2.5 mL of 1000 ppm standard to 50 mL of deionized water, a working standard of 50 ppm is prepared. Chloride, sulfate, and phosphate standards can all be combined into one working standard. 28 mM sodium hydroxide is used as eluent. Approximately 1000 ml of high purity water is added to a 2000 ml volumetric flask, 5.6 mL of 10N sodium hydroxide solution is added to the water in the flask, the total fluid volume in the flask is brought to 2000 ml with high purity water, mixed well by inversion and transferred to eluent bottle in the ICS. A high dilution is necessary for all samples in order to minimize interference from the succinate ion, which will elute using the anion columns. Without the dilution, succinate would overload the instrument. All liquid samples for testing are diluted with deionized water and filtered through 0.2 □m filter. The solid samples are diluted with deionized water and filtered through 0.2 □m filter. The following parameters are followed in running the ICS. Flow Rate: 1.5 mL/minute; Column Temperature: 30° C.; Cell Temperature: 35° C.; Suppressor Current: 104 mA; Analysis Time: 13 minutes; Sample Deliver Speed: 4 mL/minute; Delay Volume: 125 □L; Flush Factor: 5; Data Collection Rate: 5 Hz. Control sample and blank sample are run after every 10 injections and at the completion of a run to account for any possible drift. Samples and controls are integrated to calculate the results.

Analytical GC Procedures.

In order to calculate the conversion efficiency and the selectivity for various products of carbonyl reduction process involving metal catalyst, appropriate analytical procedure for the quantitative determination of various compound of interest including 2-PY, NMP, GBL, BDO and THF were established with a gas chromatographic apparatus.

Apparatus:

HP 5890 GC apparatus with FID, Capillary column RTX1701, 60 m, 0.53 mm internal diameter and film thickness of 1 □m. The following instrument conditions were used. Split vent: 100 ml/min; Air flow: 300 ml/min; Hydrogen flow: 30 ml/min; Head pressure: 10 psi; Signal range: 7; Injection volume: 1.0 □l; Initial Temperature: 40° C.; Initial Time: 6 min; Ramp Rate: 7° C./min; Final Temperature: 200° C.; Final Time: 4 min; Total Run Time: 32.85 min; Injector and detector temperature: 220° C. and 250° C.

Sample Preparation and Analysis:

50 □l of sample solution was taken and weighed on an analytical balance and 100 □l of internal standard solution comprising 1,2-propanediol in ethanol (10 mg/ml) was added to the sample solution. To this 150 □l of combined solution, 850 □l of ethanol was added. 1 □l of the resulting 1000 □l solution was injected into the GC apparatus to quantify the wt % of each of the components present in the initial sample solution. The gas chromatographic traces were integrated and the wt % of each of the components was calculated. For each hydrogenation reaction, six different samples were drawn from the Parr Reactor at specified time point. The first sample solution was drawn when the reactor was at still at the room temperature. Second sample was drawn when the temperature of the Parr Reactor reached a target reaction temperature. The third, fourth, fifth, and sixth sample solutions were drawn 120 minutes, 240 minutes, 360 minutes, and 21 hours respectively after the Parr Reactor reached the target temperature. Depending on the experiment, the target temperature was within the range of 100° C. to 240° C.

Initial hydrogenation reaction was run with a number of ‘off-the-shelf’ catalysts, such as 1% Pt/C, 5% Pd/C, 5% Ru/C, 5% Rh/C and some Mo and Cr promoted Raney-Ni catalysts. The initial process temperature was suggested to be in the range 180-225° C. at 1000 psig H₂ using 10% aqueous succinimide solution.

Catalytic Hydrogenation Reaction.

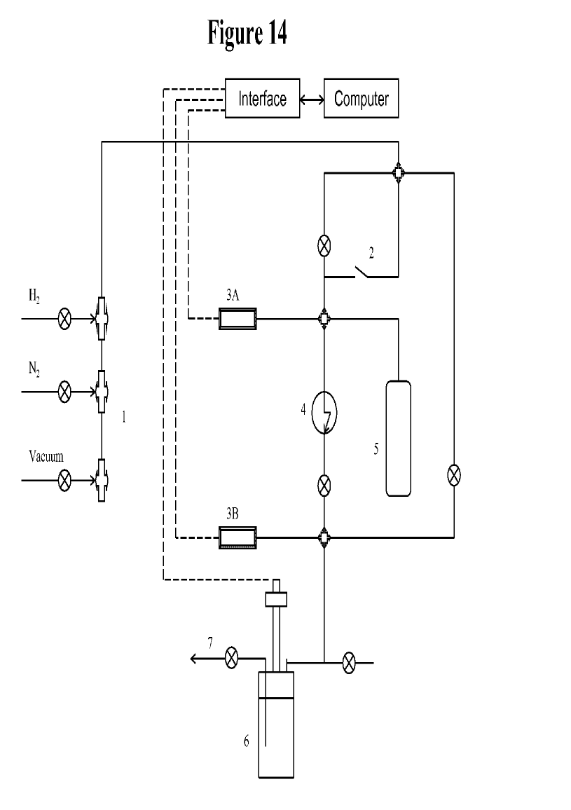

The hydrogenation runs were performed in a standard 100 mL Parr Reactor as shown in FIG. 14. The Parr Reactor unit consists of three individual sections—the feed section, the high-pressure section and low pressure section. The ports for the hydrogen, nitrogen, and vacuum line (1) are located in the feed section. The high-pressure section consisted of a forward pressure regulator (4), calibrated volume ballast reservoir (5) and pressure transducer (3).

The low-pressure section is in line with the reactor (6) and its pressure is being monitored using a pressure transducer (3a). For a typical hydrogenation run, the gas consumption in the reactor section leads to a continuous pressure drop in the calibrated ballast reservoir. That pressure, along with the pressure in the autoclave, the tachometer reading and the reaction temperature are continuously monitored and recorded at chosen pressure-drop increments. From the pressure drop, the amount of the consumed hydrogen is calculated. The high-pressure section is equipped with a solenoid valve (2), the purpose of which is to refill the ballast tank when the pressure in that flask drops below certain level. For hydrogenation reactions run in batch mode, liquid samples can be extracted at predetermined time intervals through an extraction port (7) fitted with 0.45 □m filtering element and 1/16″ needle valve attached to the dip tube within the reactor space.

General Procedure for Hydrogenation Reaction in Batch Mode.

In a representative run the reactor flask is charged with 1 gm (Dry basis) of the powdered catalyst, 5 gm of succinimide dissolved in 45 g of appropriate solvent. The system was alternately purged with nitrogen (by five pressurize-release cycles). The reactor was next flushed with five cycles of hydrogen and the pressure adjusted to 450 psi. The heating was started to the 200° C. under stirring of 250 rpm for which step takes an average of 45 min. When the temperature stabilized at this setting, the pressure in the reactor was adjusted to 1000 psi, the stirring was set to 1200 RPM and the data acquisition was initiated simultaneously. The progress of the reaction was monitored by pressure drop in the ballast reservoir and samples were extracted at predetermined time intervals for detailed kinetic analysis.

Materials.

The Succinimide was obtained from TCI (Tokyo Chemical Industry Co. Ltd., Japan with purity 98%. The solvents used for the hydrogenation reaction were used as received without further purification. The ‘off-the-shelf’ commercial catalysts used for the hydrogenation experiments are listed in the Table 2.

### Example 1

### Generation of Fermentation Broth

Fermentation broth containing diammonium succinate was generated by means of growing KJ122 strain of Escherichia coli in a minimal medium under anaerobic condition as described in the published international patent applications WO2008/115958, WO2011/115067, WO2011/063055, WO2011/063157, WO2011/082378, WO2011/123154, WO2011/130725, WO2012/018699 and WO2012/082720, all of which are incorporated herein by reference. Either dextrose or sucrose was used as the source of organic carbon.

At the end of the specified time for fermentation to achieve maximum yield for diammonium succinate, the fermentation broth was removed from the fermenter and the bacterial cells were removed by microfiltration. The clarified fermentation broth was subject to ultrafiltration to remove other macromolecules such as proteins which could interfere in further downstream chemical processing involving deammoniation, cyclization, alkylation and catalyst-mediated carbonyl reduction leading to the production of 2-pyrrolidone and N-methylpyrrolidone.

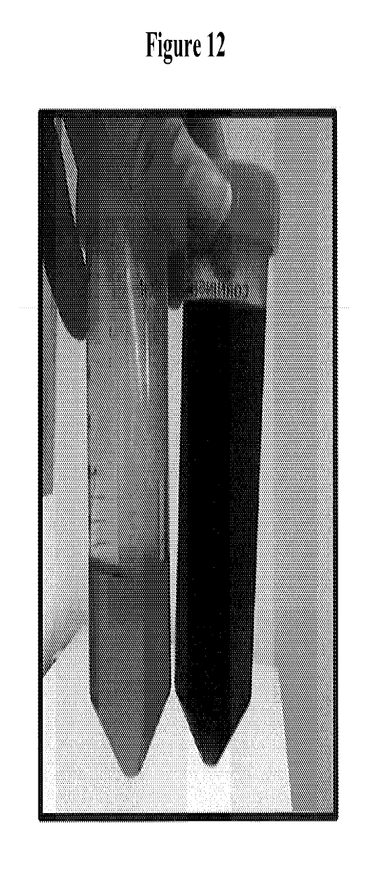

The concentration of the succinate in the fermentation broth after microfiltration and ultrafiltration was found to be 70 g/L. The fermentation broth was further concentrated in a vacuum evaporation apparatus to concentrate an aqueous portion of the broth at 65-70° C. This vacuum evaporation process increased the succinate concentration to 212 g/L. This increase in succinate concentration was accompanied by the substantial darkening of the broth (FIG. 12).

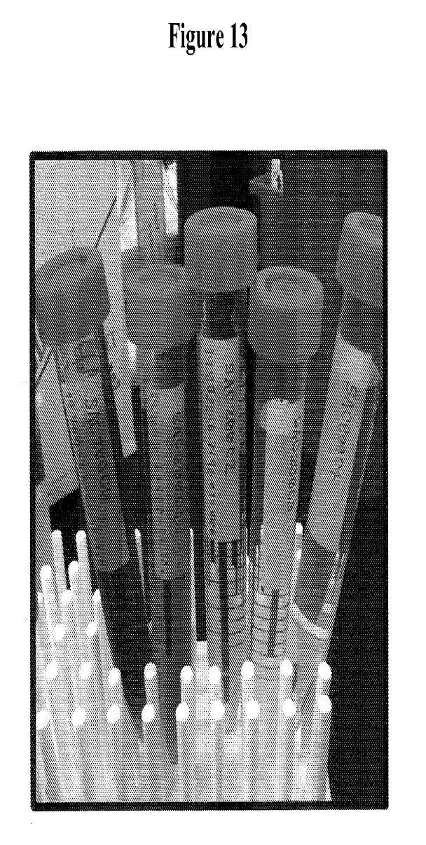

Activated carbon treatment of fermentation broth was conducted to remove the any impurities that may be present in the fermentation broth after microfiltration, ultrafiltration and vacuum concentration. Five different samples were prepared with different amounts of activated carbon (Calgon CPG LF12×40) as shown in the Table 3. The samples were thoroughly mixed and left at room temperature for an hour and the activated carbon was then removed by centrifugation. As shown in FIG. 13, with activated carbon treatment, it is possible to completely remove the coloring materials present in the concentrated broth.

### Example 2

### Aqueous-Phase Diammonium Succinate Reaction at 150° C. for 6 Hours

In order to determine the efficiency of thermochemical conversion of diammonium succinate into succinimide, aqueous-phase reaction was carried out at 150° C. with the concentrated broth after activated carbon treatment. 50 ml of broth with the succinic acid concentration of 212.36 g/L was transferred to a 75-ml Parr reactor equipped with a pressure transducer, a thermowell, and a heater block. A small magnetic stir bar was added to the reactor. The system was purged under nitrogen three times and the system was under atmospheric pressure at room temperature. The heater block was set to achieve a temperature of 150° C. The temperature was monitored using a thermocouple inserted into a thermowell. A sample was taken at 6 hours after incubation at 150° C. and analyzed using HPLC apparatus as described above. The molar concentrations of succinic acid, succinimide, succindiamide and succinamic acid were measured. As the results shown in Table 4 indicates that under the thermochemical conversion under an aqueous environment, succinamic acid was produced at a higher concentration than succinimide. Furthermore, chloride, phosphate, and sulfate ions from the feed remain soluble with the end product.

### Example 3

### Reaction of Diammonium Succinate in Diglyme at 150° C. For 6 Hours

Thermochemical conversion of diammonium succinate into succinimide, succinamic acid and succindiamide was determined in solvent environment. The concentrated fermentation broth with diammonium succinate concentration of 371.43 g/L was used in this experiment without activated carbon treatment. 150 ml of fermentation broth was transferred to a 1000 ml round-bottom flask. A small magnetic stir bar was added to the flask. 250 ml of diglyme with a normal boiling point of 162° C. was added to the flask. The flask was placed in an oil bath on the top of a hot plate. The system was purged under nitrogen three times and then the system was put under vacuum around 26 inches Hg of vacuum and the stirring rate was kept at 1000 rpm. Under the vacuum, the heat was turned on and the water in the aqueous phase was allowed to evaporate. The temperature was measured through a thermocouple. Once the evaporation of water in the aqueous phase stopped, the temperature was raised to 150° C. After 6 hours of reaction at 150° C., the product was filtered through a 2-ply filter paper to remove some suspended solids. The liquid portion was analyzed using HPLC apparatus as described above. The solid portion was washed with acetone and filtered 3 times to remove the remaining diglyme and dried in an oven at 105° C. overnight. Then the solid portion was also analyzed. The molar concentrations of succinic acid, succinimide, succindiamide and succinamic acid were measured. As the results shown in Table 5 indicates that during the thermochemical conversion under an organic solvent environment, succinimide was produced at a higher concentration than succinamic acid. Furthermore, ionic impurity like chloride, phosphate, and sulfate impurities was present in a very small amount in an organic solvent phase. The analysis of the solid phase shows that it consists of many ionic impurities that precipitate out of the solvent solution. The solid was found to contain potassium (19.6 wt %), phosphate (53.9 wt %), ammonium (2.2 wt %), and sulfate (2.0 wt %). By filtering out this solid, the succinimide solution in organic solvent is purified prior to the hydrogenation reaction.

### Example 4

### Hydrogenation Reaction with Water as the Reaction Solvent

Succinimide was obtained from TCI (Tokyo Chemical Industry Co. Ltd., Japan) with 98% purity. Five gram of succinimide (49.4 mmol) was dissolved in 45 gram of water. The hydrogenation reaction was carried out with a number of different catalysts in a Parr Reactor under hydrogen environment (1000 psi) at 200° C. as described above. For carbon supported catalysts, catalysts were used at an amount necessary to provide 0.47 mmol metal. Raney Nickel catalyst was used at 1 gram. Samples were collected at different time points and analyzed for the concentration of pyrrolidine, gammabutyrolactone, pyrrolidone and succinamic acid and the results are shown in Table 6. As the results in the Table 6 indicate, under the aqueous environment, there was significant hydrolysis of succinimide into succinamic acid and the molar selectivity for 2-pyrrolidone varied very much with different catalysts.

### Example 5

### Hydrogenation Reaction with Diglyme or Glyme as the Reaction Solvent

Succinimide was obtained from TCI (Tokyo Chemical Industry Co. Ltd., Japan) with 98% purity. Five gram of succinimide (49.4 mmol) was dissolved in 45 gram of diglyme or glyme. The hydrogenation reaction was carried out with a number of different catalysts in a Parr Reactor under hydrogen environment (1000 psi) at 200° C. as described above. For carbon supported catalysts, catalysts were used at amount necessary to provide 0.47 mmol metal. Raney Nickel catalyst was used at 1 gram. Samples were collected at different time points and analyzed for the concentration of pyrrolidine, gammabutyrolactone, pyrrolidone and succinamic acid and the results are shown in Tables 7, 8 and 9. Table 7 shows the results of the hydrogenation reaction with diglyme as the reaction solvent. Table 8 shows the results of the hydrogenation reaction with glyme as the reaction solvent. Table 9 shows the results of catalyst optimization study where diglyme was used as the reaction solvent with 5% Rh/Carbon catalyst. As the result in the Tables 7, 8 and 9 indicate, under the solvent environment, there was a significant reduction in the hydrolysis of succinimide into succinamic acid as compared to the similar reaction under aqueous environment. Furthermore, the reaction time required to achieve maximum conversion was reduced significantly compared to that in the aqueous environment.

The applicants' invention has been described in detail above with particular reference to preferred embodiment. A skilled practitioner familiar with the above detailed description can make any modification without departing from the spirit of the claims that follow.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

TABLE 1
Description of the terms used in the calculations

| | |
|---|---|
| Reactant Conversion (“Cnv_{X}”) | ${Cnv}_{X} ( \% ) = \frac{{[ X ]}_{i n} - {[ X ]}_{out}}{{[ X ]}_{i n}} \times 100$ |
| Product Selectivity (“Sel_{Y}”) | ${Sel}_{Y} ( \% ) = \frac{{[ Y ]}_{out}}{{[ X ]}_{i n} - {[ X ]}_{out}} \times 100$ |
| Product Yield | (Cnvₓ) X (Sel_{Y}) |

where:
X denotes a reactant;
Y denotes a component of the product;
[X]ᵢₙ is the mole of X in the starting composition;
[X]ₒᵤₜ is the mole of X in the exit flow; and
[Y]ₒᵤₜ is the mole of Y in the exit flow.

TABLE 2
Commercial catalysts used in the present invention

| | | | | | % |
|---|---|---|---|---|---|
| No. | Catalyst | Vendor | Type | Lot | H₂O |
| 1 | 5%Ru/C | Johnson Matthey | D101038-5 | C 5997 | 56.2 |
| 2 | 5%Rh/C | Johnson Matthey | C101038-5 | C 5306 | 51.2 |
| 3 | 5%Rh/C | Johnson Matthey | C101023-5 | C 4037 | 59.4 |
| 4 | 5%Rh/C | Johnson Matthey | | C 5444 | 66.5 |
| 5 | 5%Rh/C | Johnson Matthey | C101023-5 | C 6020 | 58.6 |
| 6 | 5%Rh/C | Johnson Matthey | | C 5322 | 61.6 |
| 7 | 5%Pd/C | Johnson Matthey | | C 5880 | |
| 8 | 5%Pd/C | Johnson Matthey | | C 5879 | 63.9 |
| 9 | 2.5% Pd/C | Johnson Matthey | | C 5887 | 66.9 |
| 10 | 10%Pd/C | Evonik | E 101 NE/W | 20089562 | |
| 11 | Ra—Ni | Grace | 2400 | | |
| 12 | Ra—Ni | Grace | 2724 | | |
| 13 | Ra—Ni | Grace | 6800 | | |
| 14 | Sponge Ni | Alfa Aesar | A 5000 | L21S013 | |
| 15 | Sponge Ni | Johnson Matthey | A 7063 | 706300697 | |
| 16 | Sponge Ni | Johnson Matthey | A 7000 | 7000001555 | |

TABLE 3
Removal of color of the concentrated broth with activated carbon

| Sample | SAC200C0 | SAC200C1 | SAC200C2 | SAC200C3 | SAC200C4 |
|---|---|---|---|---|---|
| Broth added (g) | 55.20 | 55.26 | 55.25 | 55.20 | 55.46 |
| Carbon added (g) | 0 | 0.55 | 1.65 | 2.74 | 5.53 |
| % Carbon | 0% | 0.99% | 2.9% | 4.7% | 9.1% |

TABLE 4
Aqueous-phase DAS reaction at 150° C. for 6 hours

| Compound | Feed (mmol) | Liquid Product (mmol) |
|---|---|---|
| Succinic acid | 89.9 | 64.1 |
| Succinimide | 0.0 | 11.3 |
| Succinamic acid | 0.0 | 23.1 |
| Succindiamide | 0.0 | 1.0 |
| Chloride, sulfate, phosphate | 3.6 | 3.2 |

TABLE 5
Solvent-phase DAS reaction at 150° C. for 6 hours

| Compound | Feed (mmol) | Product (mmol) |
|---|---|---|
| Succinic acid | 472 | 202 |
| Succinimide | 0.0 | 228 |
| Succinamic acid | 0.0 | 57.0 |
| Succindiamide | 0.0 | 12.3 |
| Chloride, sulfate, phosphate | 10.2 | 0.02 |

TABLE 6
Products of hydrogenation reaction in aqueous phase

| | | | | Mole Selectivity | | | |
|---|---|---|---|---|---|---|---|
| | | Time | Conversion | Pyrrolidine | GBL | Pyrrolidone | Succinamic Acid |
| File # | Catalyst | h | % | % | % | % | % |
| MYRT002 | 5% Pd/C C-5880 | 20 | 98.8 | 0.0 | 2.5 | 57.1 | 8.7 |
| | (Johnson Matthey) | | | | | | |
| MYRT004 | 5% Pd/C C-5879 | 21.5 | 98.6 | 0.0 | 1.3 | 60.0 | 1.5 |
| | (Johnson Matthey) | | | | | | |
| MYRT005 | 5% Rh/C C-5306 | 21 | 96.1 | 0.0 | 1.1 | 33.5 | 11.4 |
| | (Johnson Matthey) | | | | | | |
| MYRT006 | 2.5% Pd/C C-5887 | 21 | 100 | 0.0 | 1.0 | 47.4 | 2.0 |
| | (Johnson Matthey) | | | | | | |
| MYRT007 | 10% Pd/C- Ev | 21 | 98.4 | 0.0 | 0.8 | 50.3 | 2.4 |
| | (E-101 NE/W | | | | | | |
| MYRT010 | RaNi-2400 | 20.5 | 100.0 | 0.0 | 4.3 | 77.3 | 3.5 |
| | (Grace) | | | | | | |
| MYRT011 | Sponge Ni-A-7000 | 21 | 98.8 | 0.8 | 3.6 | 66.5 | 4.5 |
| | (Johnson Matthey) | | | | | | |
| MYRT012 | Sponge Ni A-7063 | 21 | 99.6 | 1.5 | 4.7 | 71.4 | 0.9 |
| | (Johnson Matthey) | | | | | | |

TABLE 7
Products of hydrogenation reaction with diglyme as the solvent

| | | | | Molar Selectivity | | | |
|---|---|---|---|---|---|---|---|
| | | Time | Conversion | Pyrrolidine | GBL | Pyrrolidone | Succinamic Acid |
| File # | Catalyst | (h) | % | % | % | % | % |
| MYRT017 | RaNi-2400 (Grace) | 6 | 99.7 | 2.3 | 7.5 | 75.4 | 0.0 |
| MYRT018 | RaNi-2724 (Grace) | 4 | 100 | 2.1 | 3.5 | 58.8 | 4.6 |
| MYRT019 | RaNi-6800 (Grace) | 6 | 99.3 | 2.5 | 22.9 | 70.5 | 0.3 |
| MYRT020 | RaNi-A5000 | 6 | 99.3 | 2.9 | 13.9 | 76.9 | 0.0 |
| | (Alfa Aesar) | | | | | | |
| MYRT021 | Sponge-Ni A7063 | 2 | 98.2 | 2.8 | 19.0 | 73.8 | 0.0 |
| | (Johnson Matthey) | | | | | | |
| MYRT022 | Sponge-Ni A7000 | 4 | 98.7 | 2.4 | 15.8 | 68.0 | 5.7 |
| | (Johnson Matthey) | | | | | | |
| MYRT024 | 5% Ru/C C5997 | 4 | 100.0 | 0.9 | 1.7 | 67.8 | 0.7 |
| | (Johnson Matthey) | | | | | | |
| MYRT025 | 5% Rh/C C5306 | 21 | 81.2 | 3.3 | 1.9 | 87.4 | 0.0 |
| | (Johnson Matthey) | | | | | | |

TABLE 8
Products of hydrogenation reaction with glyme as the solvent

| | | | | Mole Selectivity | | | |
|---|---|---|---|---|---|---|---|
| | | | | | | | Succinamic |
| | | Time | Conversion | Pyrrolidine | GBL | Pyrrolidone | Acid |
| File # | Catalyst | h | (%) | (%) | (%) | (%) | (%) |
| MYRT030 | RaNi-2400 (Grace) | 4 | 99.1 | 0.0 | 19.0 | 69.2 | 0.0 |
| MYRT031 | RaNi-6800 (Grace) | 4 | 90.1 | 0.0 | 16.5 | 73.6 | 0.0 |
| MYRT039 | Sponge Ni A5000 (Alfa Aesar) | 6 | 100.0 | 0.0 | 24.0 | 78.0 | 0.0 |
| MYRT032 | Sponge-Ni A7063 | 4 | 100.0 | 0.0 | 13.6 | 74.3 | 0.0 |
| | (Johnson Matthey)) | | | | | | |
| MYRT040 | Sponge A7000 | 6 | 100.0 | 0.0 | 16.7 | 74.5 | 0.0 |
| | (Johnson Matthey) | | | | | | |
| MYRT043 | 5% Rh/C C5306 | 21 | 94.8 | 1.3 | 0.8 | 90.9 | 0.0 |
| | (Johnson Matthey) | | | | | | |

TABLE 9
Products of hydrogenation reaction with diglyme as the reaction solvent and
5% Rh/Carbon catalysts (Catalyst Optimization Study)

| | | | Catalyst | | | | Mole Selectivity | | | |
|---|---|---|---|---|---|---|---|---|---|---|
| | T | P | | Mass | Time | Con- | Pyrrolidine | GBL | Pyrrolidone | Succinamic |
| File # | oC | psi | Type | G | h | version % | % | % | % | Acid % |
| MYRT025 | 200 | 1000 | JM C5306 | 1.0 | 21 | 81.2 | 3.3 | 1.9 | 87.4 | 0.0 |
| MYRT036 | 220 | 1000 | JM C5306 | 1.0 | 21.5 | 98.5 | 0.7 | 1.7 | 86.0 | 0.0 |
| MYRT049 | 200 | 1000 | JM C5306 | 2.0 | 6 | 92.3 | 0.4 | 0.7 | 78.2 | 0.0 |
| MYRT051 | 200 | 1000 | JM C4037 | 1.0 | 6 | 68.9 | 0.0 | 1.3 | 80.6 | 1.6 |
| MYRT050 | 200 | 1000 | JM C5444 | 1.0 | 6 | 78.9 | 0.0 | 0.9 | 81.0 | 0.0 |
| MYRT059 | 210 | 1000 | JM C5444 | 1.0 | 6 | 96.1 | 0.6 | 0.9 | 80.3 | 1.4 |
| MYRT053 | 220 | 1000 | JM C5444 | 1.0 | 6 | 99.3 | 0.4 | 0.9 | 81.8 | 3.4 |
| MYRT054 | 200 | 1400 | JM C5444 | 1.0 | 6 | 98.5 | 0.4 | 1.2 | 82.4 | 0.7 |
| MYRT052 | 200 | 1000 | JM C5444 | 1.5 | 6 | 97.0 | 0.9 | 0.8 | 84.8 | 1.4 |
| MYRT060 | 200 | 1000 | JM C6020 | 1.0 | 6 | 77.8 | 0.0 | 0.7 | 88.9 | 0.5 |
| MYRT065 | 200 | 1400 | JM C6020 | 1.0 | 6 | 77.4 | 0.5 | 1.1 | 90.7 | 0.0 |
| MYRT061 | 200 | 1000 | JM C5322 | 1.0 | 6 | 70.3 | 0.6 | 0.0 | 80.4 | 0.9 |

### REFERENCES

U.S. Pat. No. 3,080,377
U.S. Pat. No. 3,198,808
U.S. Pat. No. 3,448,118
U.S. Pat. No. 3,681,387
U.S. Pat. No. 3,812,148
U.S. Pat. No. 3,910,951
U.S. Pat. No. 4,263,175
U.S. Pat. No. 4,356,124
U.S. Pat. No. 4,841,069
U.S. Pat. No. 4,904,804
U.S. Pat. No. 5,101,045
U.S. Pat. No. 5,157,127
U.S. Pat. No. 5,434,273
U.S. Pat. No. 6,603,021
U.S. Pat. No. 6,632,951
U.S. Pat. No. 6,670,483
U.S. Pat. No. 6,706,893
U.S. Pat. No. 7,199,250
U.S. Pat. No. 7,674,916
U.S. Pat. No. 7,973,177
U.S. Pat. No. 8,017,790
U.S. Pat. No. 8,084,626
U.S. Pat. No. 8,193,375
U.S. Pat. No. 8,203,021
U.S. Pat. No. 8,246,792
U.S. Patent Application Publication No. US 2004/0176589
U.S. Patent Application Publication No. US 2010/0044626
U.S. Patent Application Publication No. US 2010/184171
U.S. Patent Application Publication No. US 2011/0237831
U.S. Patent Application Publication No. US 2011/0245514
U.S. Patent Application Publication No. US 2011/0245515
U.S. Patent Application Publication No. US 2011/0272269
U.S. Patent Application Publication No. US 2011/0297527
U.S. Patent Application Publication No. US 2011/0301364
U.S. Patent Application Publication No. US 2011/0306777
International Patent Application Publication No. WO2008/115958
International Patent Application Publication No. WO2011/063055
International Patent Application Publication No. WO2011/063157
International Patent Application Publication No. WO2011/082378
International Patent Application Publication No. WO2002/102772
International Patent Application Publication No. WO2010/115067
International Patent Application Publication No. WO2011/119427
International Patent Application Publication No. WO2011/123154
International Patent Application Publication No. WO2011/123269
International Patent Application Publication No. WO 2011/130725
International Patent Application Publication No. WO2011/123270
International Patent Application Publication No. WO2011/146449
International Patent Application Publication No. WO2011/146561
International Patent Application Publication No. WO2012/018699
International Patent Application Publication No. WO2012/082720
Corma, A., Iborra, S., Velty, A. (2007) Chemical routes for the transformation of biomass into chemicals. Chem. Rev. 107: 2411-2502.
Davis, S. 1,4-Butanediol, CEH Market Research Report, October 2010.
Delhomme, C., Weuster-Botz, D., Kuhn, F. E. (2009) Succinic acid from renewable resources ad a C4 building-block chemical—A review of the catalytic possibilities in aqueous media. Green Chem 11: 13-26.
Frye, Jr, J. G., Zacher, A. H., Werpy, T. A., and Wang, Y. (2005) Catalytic preparation of pyrrolidones from renewable resources. Catalysis of Organic Reactions, J. R. Sowa, Jr (editor), CRC Press, pp 145-154.
Jantama, K., Haupt, M. J., Svoronos, S. A., Zhang, X., Moore, J. C., Shanmugham, K. T., Ingram, L. O. (2008) Combining metabolic engineering and metabolic evolution to develop nonrecombinant strains of E. coli C that produce succinate and malate. Biotech Bioeng 99: 1140-1153.
Jantama, K., Zhang, X., Moore, J. C., Shanmugham, K. T., Svoronos, S. A., Ingram, L. O. (2008) Eliminating side products and increasing succinate yields in engineered strains of Escherichia coli C. Biotech. Bioeng. 101: 881-893.
2-Pyrrolidone report, U.S. EPA HPV Challenge Program, Dec. 30, 2002. (online access: http://www.epa.gov/hpv/pubs/summaries/2pyrroli/c14223tp.pdf).
Reisch, M. (2008) Chemical and Engineering News: Vol. 86, Issue 29, 2008.
Ullmann's Encyclopedia of Industrial Chemistry, Wiley-VHC Verlab GmbH, 2002.
Varadarajan, S. and Miller, D. J. (1999) Catalytic upgrading of fermentation-derived organic acids. Biotechnol Prog 15: 845-854.
Zhang, X., Jantama, K., Moore, J. C., Shanmugam, K. T., Ingram, L. O. (2009) Metabolic evolution of energy-conserving pathways for succinate production in Escherichia coli. Proc. Natl. Acad. Sci. U.S.A. 106: 20180-20185.

What is claimed is:

1. A process for preparing succinimide comprising the steps of:
(a) providing a fermentation broth comprising diammonium succinate;
(b) adding a polar organic solvent with boiling point higher than that of water to said fermentation broth;
(c) evaporating water in said fermentation broth;
(d) raising temperature of said fermentation broth to at least 120° C. to convert said diammonium succinate to succinimide.

2. The Process according to claim 1, wherein said fermentation broth is a concentrated fermentation broth.

3. The process according to claim 1, wherein said fermentation broth is subjected to ultrafiltration process before converting diammonium succinate to succinimide.

4. The process according to claim 1, wherein said fermentation broth is subjected to adsorption process to remove sugars and amino acids in the fermentation broth.

5. The process according to claim 1, wherein said polar organic solvent is selected from a group consisting of diglyme, triglyme, tetraglyme, propylene glycol, dimethylsulfoxide, dimethylformamide, dimethylacetamide, dimethylsulfone, sulfolane, polyethylene glycol, butoxytriglycol, N-methylpyrrolidone, 2-pyrrolidone, gammabutyrolactone, dioxane and methyl isobutyl ketone.

6. The process according to claim 1 wherein said polar organic solvent is diglyme.

7. A process for preparing 2-pyrrolidone comprising the steps of:
(a) providing a fermentation broth comprising diammonium succinate;
(b) adding a polar organic solvent with boiling point higher than that of water to said fermentation broth;
(c) evaporating water in said fermentation broth;
(d) raising temperature of said fermentation broth to at least 120° C. to convert said diammonium succinate to succinimide.
(e) hydrogenating said succinimide in the presence of a catalyst in a solvent phase to produce 2-pyrrolidone; and
(f) recovering 2-pyrrolidone by distillation.

8. The Process according to claim 7, wherein said fermentation broth is a concentrated fermentation broth.

9. The process according to claim 7, wherein said fermentation broth is subjected to ultrafiltration process before converting diammonium succinate to succinimide.

10. The process according to claim 7, wherein said fermentation broth is subjected to adsorption process to remove sugars and amino acids in the fermentation broth.

11. The process according to claim 7, wherein said polar organic solvent is selected from a group consisting of diglyme, triglyme, tetraglyme, propylene glycol, dimethylsulfoxide, dimethylformamide, dimethylacetamide, dimethylsulfone, sulfolane, polyethylene glycol, butoxytriglycol, N-methylpyrrolidone, 2-pyrrolidone, gammabutyrolactone, dioxane and methyl isobutyl ketone.

12. The process according to claim 7 wherein said polar organic solvent is diglyme.

13. A process for preparing N-methylsuccinimide comprising the steps of:
(a) providing a fermentation broth comprising diammonium succinate;
(b) adding a polar organic solvent with boiling point higher than that of water and methanol to said fermentation broth;
(c) converting said diammonium succinate to N-methylsuccinimide;
(d) recovering said N-methylsuccinimide in an organic solvent;

14. The process according to claim 13, wherein said fermentation broth is a concentrated fermentation broth.

15. The process according to claim 13, wherein said fermentation broth is subjected to ultrafiltration process before converting diammonium succinate to n-methylsuccinimide.

16. The process according to claim 13, wherein said fermentation broth is subjected to adsorption process to remove sugars and amino acids in the fermentation broth.

17. The process according to claim 13, wherein said polar organic solvent is selected from a group consisting of diglyme, triglyme, tetraglyme, propylene glycol, dimethylsulfoxide, dimethylformamide, dimethylacetamide, dimethylsulfone, sulfolane, polyethylene glycol, butoxytriglycol, N-methylpyrrolidone, 2-pyrrolidone, gammabutyrolactone, dioxane and methyl isobutyl ketone.

18. The process according to claim 13 wherein said polar organic solvent is diglyme.

19. A process for preparing N-methylpyrrolidone comprising the steps of:
(a) providing a fermentation broth comprising diammonium succinate;
(b) adding a polar organic solvent with boiling point higher than that of water and methanol to said fermentation broth;
(c) evaporating water in said fermentation broth;
(d) raising temperature of said fermentation broth to at least 120° C. to convert said diammonium succinate to N-methylsuccinimide;
(e) hydrogenating said N-methylsuccinimide in the presence of a catalyst in a solvent phase to produce N-methylpyrrolidone; and
(f) recovering N-methylpyrrolidone by distillation.

20. The process according to claim 19, wherein said fermentation broth is a concentrated fermentation broth.

21. The process according to claim 19, wherein said fermentation broth is subjected to ultrafiltration process before converting diammonium succinate to N-methylsuccinimide.

22. The process according to claim 19, wherein said fermentation broth is subjected to adsorption process to remove sugars and amino acids in the fermentation broth.

23. The process according to claim 19, wherein said polar organic solvent is selected from a group consisting of diglyme, triglyme, tetraglyme, propylene glycol, dimethylsulfoxide, dimethylformamide, dimethylacetamide, dimethylsulfone, sulfolane, polyethylene glycol, butoxytriglycol, N-methylpyrrolidone, 2-pyrrolidone, gammabutyrolactone, dioxane and methyl isobutyl ketone.

24. The process according to claim 19 wherein said polar organic solvent is diglyme.
